(19) United States
(12) Patent Application Publication
(10) Pub. No.: US 2007/0038298 A1
(43) Pub. Date: Feb. 15, 2007

(54) Repair of tympanic membrane using placenta derived collagen biofabric

(76) Inventors: Joseph W. Sulner, Paramus, NJ (US); Janice Smiell, Morristown, NJ (US); Sharon L. Bourke, Piscataway, NJ (US); Patricia A. Murphy, Hillsborough, NJ (US)

Correspondence Address: JONES DAY, 222 EAST 41ST ST, NEW YORK, NY, 10017, US

(21) Appl. No.: 11/479,801

(22) Filed: Jun. 29, 2006

Related U.S. Application Data
(60) Provisional application No. 60/696,167, filed on Jun. 30, 2005.

(51) Int. Cl. A61F 2/18

(52) U.S. Cl. 623/10; 623/23.72

(57) ABSTRACT

The present invention provides a method of repairing a tympanic membrane deformity, such as a tympanic membrane perforation, commonly referred to as tympanoplasty or myringoplasty, using a collagen biofabric. The collagen biofabric is preferably laminated. The invention further provides kits comprising one or more pieces of collagen biofabric, for example laminated collagen biofabric, for the repair of a tympanic membrane.

This application claims benefit of U.S. Provisional Application No. 60/696,167, filed Jun. 30, 2005, which is hereby incorporated by reference herein in its entirety.

### 1. FIELD OF THE INVENTION

The present invention relates to the repair of the tympanic membrane, commonly referred to as tympanoplasty or myringoplasty, using a collagen biofabric.

### 2. BACKGROUND OF THE INVENTION

The first component of the middle ear to receive sound waves is the tympanic membrane, also known as the eardrum. Sound waves striking the tympanic membrane are transmitted through a series of tiny bones—the malleus, incus and stapes—to the cochlea, where the sound waves are sensed and processed. The tympanic membrane itself is living tissue.

Tympanic membrane deformities, such as perforations, interfere with the transmission and perception of sound. Perforations are usually caused by trauma or infection. Examples of traumatic causes of perforated eardrums include open hand blows to the ear (i.e., boxing the ears); skull fractures; sudden explosions; objects such as a bobby pin or cotton swab pushed too far into the ear canal; hot slag from welding or acid entering the ear canal, and other traumas. Middle ear infections can cause spontaneous rupture (tear) of the eardrum, resulting in a perforation. In this circumstance, called otitis media with perforation, there may be infected or bloody drainage from the ear. A hole in the tympanic membrane may also be caused by surgical procedures, such as tympanotomy or myringotomy. A small hole may remain in the eardrum after a previously placed pressure equalization tube either falls out or is removed by the physician.

Whatever the cause of the deformity of the tympanic membrane, however, repair of the membrane is desirable. Repair of tympanic membrane perforations is accomplished, generally, in a procedure known as tympanoplasty or myringoplasty. The two are similar; however, aside from repair of the tympanic membrane itself, tympanoplasty additionally implies remediation of pathology or pathologies of the middle ear cleft, such as chronic infection, choleastoma, or ossicular chain problems. Typically, in tympanoplasty or myringoplasty, the hole in the tympanic membrane is repaired by means of a graft. Typical graft materials have, to date, included natural materials such as temporalis fascia, tragal perichondrium, skin, periosteum, loose overlay tissue, fat, vein tissue, human amniotic membrane, and homologous dura; and non-natural materials such as silastic, paper and teflon sheets. Aside from repair of the tympanic membrane, one main purpose of tympanoplasty is the creation of a middle ear space that contains air. Given this purpose, it is important that the material used to repair the tympanic membrane resists, or displays a low proclivity for, forming adhesions or promoting infection.

Generally, a perforated tympanic membrane is treated as follows. Working with a microscope, the edges of the eardrum are debrided to freshen the edges to stimulate growth, and then the occluding material, generally a thin patch or graft, is placed over the eardrum perforation so as to overlap onto the intact portions of the tympanic membrane. Commonly, the patch is a small section of cigarette paper, which is thought to provide a stent for the ingrowth of epithelial cells to fill the perforation. Usually, after closure of the tympanic membrane, hearing improvement is noted. Several applications of a patch (up to three or four) may be required before the perforation closes completely. If a paper patch does not provide prompt or adequate closure of the hole in the eardrum, or if attempts with paper patching are not successful, surgery, for example, myringoplasty or tympanoplasty, is considered. Not all otolaryngologists, however, agree that placement of a paper patch on a perforated tympanic membrane is an adequate treatment, however, citing a relatively high failure rate.

### 3. SUMMARY OF THE INVENTION

The present invention provides methods and compositions for repair of tympanic membranes. For example, the present invention provides methods and compositions for repair of a tympanic membrane injury or deformity. In one embodiment, the present invention provides a method of repairing a perforated tympanic membrane, comprising contacting said tympanic membrane with a collagen biofabric. In another specific embodiment, said contacting is sufficient to occlude the perforation. In a more specific embodiment, said perforation is a central perforation. In another more specific embodiment, said perforation is a marginal perforation. In another specific embodiment, said perforation has not healed spontaneously within two months of developing the perforation. In a specific embodiment, the proteins making up the collagen biofabric substantially retain their native conformations, e.g., the collagen biofabric is not protease-treated. In another specific embodiment, the proteins of said collagen biofabric are not cross-linked, e.g., the collagen biofabric is not fixed. In another specific embodiment, the collagen biofabric is substantially dry prior to said contacting, that is, comprises about 20% or less water by weight. In another specific embodiment, said collagen biofabric is a single layer. In another specific embodiment, said collagen biofabric is a laminate of two or more layers. In another specific embodiment, said collagen biofabric is trimmed prior to said contacting. In another specific embodiment, said collagen biofabric is about 2×2 cm prior to trimming. In another specific embodiment, said collagen biofabric is about 3×3 cm prior to trimming. In another specific embodiment, said collagen biofabric is about 2×3 cm prior to trimming. In another specific embodiment, said collagen biofabric is hydrated prior to contacting with the tympanic membrane. In another specific embodiment, the collagen biofabric is between about 2 micrometers and about 150 micrometers in thickness in the dry state. In a more specific embodiment, said biofabric is about 10 to about 50 microns in thickness in the dry state. In another specific embodiment, the biofabric is about 40 to about 50 microns in thickness in the dry state. In a more specific embodiment, the collagen biofabric that is between about 2 micrometers to about 150 micrometers in thickness in the dry state is a laminate of two or more layers. In the above embodiments, the ranges indicate average thicknesses, and are not absolute limits to thickness. In another embodiment, said collagen biofabric is contacted with the tympanic membrane through use of an applicator. In another embodiment, the invention provides one or more sheets of collagen biofabric in a sterile double-peel pouch.

3.1 Definitions

As used herein, “collagen biofabric” generally means a collagen-containing, placenta-derived amniotic or chorion membrane material that can be used as a film or sheet. A preferred collagen biofabric is the vacuum-dried, non-fixed, non-protease-treated amniotic membrane material described in Hariri, U.S. Application Publication U.S. 2004/0048796, which is hereby incorporated in its entirety, and that is produced by the methods described therein, and herein (see Examples 1, 2). The collagen biofabric is preferably made from the amnion, but may be made from the chorion, or both amnion and chorion.

As used herein, the term “bioactive compound” means any compound or molecule that causes a measurable effect on one or more biological systems in vitro or in vivo.

### 4. DETAILED DESCRIPTION OF THE INVENTION

The present invention provides methods of repairing a tympanic membrane deformity, and, more specifically, of performing a tympanoplasty or myringoplasty, using a collagenous amniotic and/or chorionic membrane material, herein referred to as a collagen biofabric.

4.1 Repair of Tympanic Membrane Using Collagen Biofabric

The present invention provides a method for the repair of a tympanic membrane using a collagen biofabric. In one embodiment, the tympanic membrane to be repaired has a deformity. The deformity may be naturally-occurring, for example, as the result of disease or infection, or may be an injury. In various embodiments, the deformity can be, for example, a perforation, e.g., an acute perforation or a chronic perforation (a perforation lasting longer than, for example, 2 months), partial or total loss of collagen in the tympanic membrane, partial or total loss of normal tympanic membrane stiffness, an atelectatic tympanic membrane (i.e., tympanic membrane in which the natural collagenous layer that stiffens the membrane is lost partially or totally), a deformity relating to cholesteatoma or tumor involvement of the middle ear, a disease of the tympanic membrane such as dimeric drum, a retraction, a retraction pocket (i.e., pocket formed in the eardrum resulting from retraction of the tympanic membrane into the middle ear cavity due to loss of pressure in the middle ear cavity), or tympanosclerosis, and the like.

Repair of a tympanic membrane deformity may, for example, encompass contacting the tympanic membrane with a collagen biofabric for a time sufficient to heal the tympanic membrane deformity, for a time sufficient to measurably improve one or more aspects of the tympanic membrane deformity, or for a time sufficient to lessen the worsening of one or more aspects of the tympanic membrane deformity, as compared to a tympanic membrane not contacted with a collagen biofabric.

As used herein, “aspects of a tympanic membrane deformity” encompasses objectively measurable criteria, such as ability of the tympanic membrane to transmit sound, hearing loss in decibels, appearance of the tympanic membrane or surrounding tissue, ingrowth of epithelial tissue into or around a perforation in the tympanic membrane, etc., or subjective criteria, such as a sense of improved hearing, lessening of discomfort or pain, etc.

In one embodiment, the deformity is a perforation. Such a perforation may, for example, be caused accidentally, by trauma, by infection, or may be caused deliberately, for example, a perforation caused by insertion of one or more tubes allowing drainage of fluids in the middle ear past the tympanic membrane and out the auditory canal (e.g., perforation(s) to allow a myringotomy tube installation, or a perforation caused by surgical removal of diseased or damaged tissue). The perforation may be acute, or the perforation may be chronic, that is, has been in existence for two months or more.

In one embodiment of repairing a tympanic membrane, a tympanic membrane having a perforation is contacted with a collagen biofabric such that the collagen biofabric partially or totally occludes the perforation. The perforation to be occluded may be a central perforation, that is, a perforation of any size that does not involve the margin of the tympanic membrane (i.e., the periphery seated in the auditory canal), or a marginal perforation (i.e., a perforation touching upon, or largely involving, the margin of the tympanic membrane). In another embodiment, only the tympanic membrane is perforated, and no other ear structure is perforated or damaged. In another embodiment, occlusion of the perforation is an adjunct to at least one other surgical procedure involving the outer, middle, or inner ear. In another embodiment, the repair of the tympanic membrane is a tympanoplasty. In another embodiment, the repair of the tympanic membrane is a myringoplasty.

The benefits of closing a tympanic membrane perforation include prevention of water entering the ear while showering, bathing or swimming (which could cause ear infection), improved hearing, and diminished tinnitus. It also might prevent the development of cholesteatoma (skin cyst in the middle ear), which can cause chronic infection and destruction of ear structures.

Tympanoplasty and myringoplasty are generally outpatient procedures. The otolaryngologist may approach repair of a tympanic membrane perforation either through the auditory canal (trans-canal approach), or via a post-auricular incision followed by folding the ear forward to expose the tympanic membrane (post-auricular approach).

Before attempting any correction of the perforation, a hearing test is generally performed, and the patient is evaluated for Eustachian tube function, as partial or complete loss of Eustachian tube function can exacerbate a tympanic membrane puncture and interfere with the adherence of a graft to the tympanic membrane. Repair of a perforated tympanic membrane generally comprises placing an occluding material on the membrane. The patient is evaluated for complications, such as extension of squamous epithelium through the perforation and into the middle ear space. In such instances, tympanoplasty or myringoplasty is preferably accompanied, where possible, by remediation of the complication.

The present invention encompasses repair of a tympanic membrane with collagen biofabric either as a first or subsequent therapy. That is, the collagen biofabric may be used to repair a tympanic membrane deformity, such as a perforation, before other remedial measures are tried. Alternatively, repair of a tympanic membrane with collagen biofabric may be performed after one or more other remedial measures have been tried and failed.

In one embodiment, repair of a tympanic membrane with collagen biofabric may additionally comprise applying an anti-infective agent to the graft and/or surrounding ear canal. Thus, in one embodiment, the invention provides a method of repairing a tympanic membrane comprising contacting the tympanic membrane with collagen biofabric and an anti-infective agent. The anti-infective agent can be contacted either prior to, concurrently with, or subsequent to contacting the tympanic membrane with the collagen biofabric. The anti-infective agent can be present separate from, or as an integral part of, the collagen biofabric. For example, the anti-infective agent can be present on the surface of the collagen biofabric, or can be impregnated in the collagen biofabric. In a specific example, the anti-infective agent is an antibiotic, a bacteriostatic agent, antiviral compound, a virustatic agent, antifungal compound, a flngistatic agent, or an antimicrobial compound. In a specific embodiment, the anti-infective agent is ionic silver. In a more specific embodiment, the ionic silver is contained within a hydrogel. Ionic silver hydrogel is a preferred anti-infective agent because it is broad spectrum, with no known bacterial resistance; its application and removal are pain-free, and the hydrogel supports autolytic debridement. In a preferred embodiment, the collagen biofabric is impregnated with silver ions prior to application to the tympanic membrane. In another embodiment, the collagen biofabric is impregnated with silver ions after application of the biofabric to the tympanic membrane, for example, by application of ear drops.

The invention further provides a method of repairing a tympanic membrane comprising contacting the tympanic membrane with collagen biofabric and a plurality of stem or progenitor cells. Preferred stem cells include, for example, mesenchymal stem cells and the placenta-derived stem cells disclosed in United States Application Publication Nos. US 2003/0032179 and US 2003/0180269 US, each of which is hereby incorporated in its entirety herein. In one embodiment, the collagen biofabric may be contacted with the stem or progenitor cells prior to contacting the tympanic membrane with the collagen biofabric. For example, collagen biofabric may be prepared prior to application on the tympanic membrane by disposing stem or progenitor cells on the surface of, or within, the collagen biofabric and allowing the stem or progenitor cells sufficient time to attach to the collagen biofabric. The stem or progenitor cells can, for example, be disposed onto the surface of, or within, the collagen biofabric at least, or no more than, 30 minutes, 1, 2, 3, 4, 5, 6, 7, 8, 9, 10, 12, 14, 16, 18, 20, 22, or 24 hours prior to application of the collagen biofabric onto the tympanic membrane. In another embodiment, the collagen biofabric may be contacted with the stem or progenitor cells after application of the collagen biofabric to the tympanic membrane. In another embodiment, a the invention provides a method of treating a tympanic membrane comprising contacting the tympanic membrane with a plurality of stem or progenitor cells, and contacting the tympanic membrane with collagen biofabric so that the collagen biofabric covers the tympanic membrane and stem or progenitor cells.

The number of stem or progenitor cells disposed onto the tympanic membrane, or onto the surface of the collagen biofabric, in any embodiment may vary, but may be at least 1×10⁶, 3×10⁶, 1×10⁷, 3×10⁷, 1×10⁸, 3×10⁸, 1×10⁹, 3×10⁹, 1×10¹⁰, 3×10¹⁰, 1×10¹¹, 3×10¹¹, or 1×10¹²; or may be no more than 1×10⁶, 3×10⁶, 1×10⁷, 3×10⁷, 1×10⁸, 3×10⁸, 1×10⁹, 3×10⁹, 1×10¹⁰, 3×10¹⁰, 1×10¹¹, 3×10¹¹, or 1×10¹²; stem or progenitor cells. Thus, in specific embodiments, the invention provides a method of repairing a tympanic membrane comprising contacting said tympanic membrane with, in either order, (a) collagen biofabric, and (b) a plurality of stem or progenitor cells comprising 1×10⁶, 3×10⁶, 1×10⁷, 3×10⁷, 1×10⁸, 3×10⁸, 1×10⁹, 3×10⁹, 1×10¹⁰, 3×10¹⁰, 1×10¹¹, 3×10¹¹, or 1×10¹²; stem or progenitor cells. In other specific embodiments, the invention provides a method of treating a tympanic membrane comprising contacting the tympanic membrane, in either order, (a) collagen biofabric, and (b) a plurality of stem or progenitor cells comprising no more than 1×10⁶, 3×10⁶, 1×10⁷, 3×10⁷, 1×10⁸, 3×10⁸, 1×10⁹, 3×10⁹, 1×10¹⁰, 3×10¹⁰, 1×10¹¹, 3×10¹¹, or 1×10¹²; stem or progenitor cells. In a more specific embodiment, said plurality of stem cells comprises two or more different stem or progenitor cell types.

The invention further provides that the use of collagen biofabric to repair a tympanic membrane deformity may be the sole treatment of the tympanic membrane, or may be in addition to another therapies or treatment used simultaneously in the course of treating or repairing a tympanic membrane. For example, the invention provides for the repair of a tympanic membrane comprising contacting the tympanic membrane with a collagen biofabric, and treating the tympanic membrane using an additional therapy not comprising contacting the tympanic membrane with a collagen biofabric, where the contacting and the additional therapy individually or together cause a measurable improvement in, maintenance of, or lessening of the worsening of, at least one aspect of a tympanic membrane deformity, as compared to a tympanic membrane not contacted with a collagen biofabric.

The invention further provides for the use of collagen biofabric to repair an ear condition in conjunction with repair of a tympanic membrane. For example, the collagen biofabric can be used to reconstruct or repair the outer or middle ear structures, including the auditory canal and middle ear chamber. The collagen biofabric, for example, may be used to repair or line the mastoid cavity, particularly where mastoid reconstruction is indicated in addition to tympanoplasty. In one embodiment, the collagen biofabric may be used to line the mastoid cavity where the mastoid cavity comprises exposed bone, that is, bone with no covering epithelial cell layer. In another embodiment, the collagen biofabric may be used as a oval window graft in stapes surgery, either alone or in conjunction with tympanoplasty or myringoplasty.

4.2 Collagen Biofabric
4.2.1 Description

The collagen biofabric used to repair a tympanic membrane may be derived from the amniotic membrane of any mammal, for example, equine, bovine, porcine or catarrhine sources, but is most preferably derived from human placenta. In a preferred embodiment, the collagen biofabric is substantially dry, i.e., is 20% or less water by weight. In another preferred embodiment, the collagen biofabric retains the native tertiary and quaternary structure of the amniotic membrane, i.e., has not been protease-treated. In another preferred embodiment, the collagen biofabric contains no collagen and other structural proteins that have been artificially crosslinked, e.g., chemically crosslinked, that is, the preferred collagen biofabric is not fixed. A preferred collagen biofabric is the dried, non-fixed, non-protease-treated amniotic membrane material described in Hariri, U.S. Application Publication U.S. 2004/0048796, which is hereby incorporated in its entirety, and that is produced by the methods described therein, and herein (see Examples 1, 2). However, the methods of the present invention can utilize any placenta-derived collagen material made by any procedure.

In a preferred embodiment, the collagen biofabric used in the treatment or repair of a tympanic membrane is translucent. In other embodiments, the collagen biofabric is opaque, or is colored or dyed, e.g., permanently colored or dyed, using a medically-acceptable dyeing or coloring agent; such an agent may be adsorbed onto the collagen biofabric, or the collagen biofabric may be impregnated or coated with such an agent. In this embodiment, any known non-toxic, non-irritating coloring agent or dye may be used.

When the collagen biofabric is substantially dry, it is about 0.1 g/cm² to about 0.6 g/cm². In a specific embodiment, a single layer of the collagen biofabric is at least 2 microns in thickness. In another specific embodiment, a single layer of the collagen biofabric used to repair a tympanic membrane is approximately 10-40 microns in thickness, but may be approximately 2-150, 2-100 microns, 5-75 microns or 7-60 microns in thickness in the dry state.

In one embodiment, the collagen biofabric is principally comprised of collagen (types I, III and IV; about 90% of the matrix of the biofabric), fibrin, fibronectin, elastin, and may further comprise glycosaminoglycans and/or proteoglycans. In certain embodiments, the collagen biofabric can comprise non-structural components, such as, for example, one or more growth factors, e.g., platelet-derived growth factors (PDGFs), vascular-endothelial growth factor (VEGF), fibroblast growth factor (FGF) and transforming growth factor-β1. The composition of the collagen biofabric may thus be ideally suited to encourage the migration of fibroblasts and macrophages, and thus the promotion of wound healing.

The collagen biofabric may be used in a single-layered format, for example, as a single-layer sheet or an un-laminated membrane. Alternatively, the collagen biofabric may be used in a double-layer or multiple-layer format, e.g., the collagen biofabric may be laminated. Lamination can provide greater stiffness and durability during the healing process. The collagen biofabric may be, for example, laminated as described below (see Section 4.2.7).

The collagen biofabric may further comprise collagen from a non-placenta source. For example, one or more layers of collagen biofabric may be coated or impregnated with, or layered with, purified extracted collagen. Such collagen may be obtained, for example, from commercial sources, or may be produced according to known methods, such as those disclosed in U.S. Pat. Nos. 4,420,339, 5,814,328, and 5,436,135, the disclosures of which are hereby incorporated by reference.

The collagen biofabric used to repair a tympanic membrane may comprise one or more compounds or substances that are not present in the placental material from which the collagen biofabric is derived. The collagen biofabric can comprise non-naturally-occuriing amounts of one or more compounds or substances that are normally present in the placental material from which the collagen biofabric is derived. For example, the collagen biofabric may be impregnated with a bioactive compound, such as those listed in Section 4.2.2, below. Such bioactive compounds include, but are not limited to, small organic molecules (e.g., drugs), antibiotics (such as Clindamycin, Minocycline, Doxycycline, Gentamycin), hormones, growth factors, anti-tumor agents, anti-flugal agents, anti-viral agents, pain medications, anti-histamines, anti-inflammatory agents, anti-infectives including but not limited to silver (such as silver salts, including but not limited to silver nitrate and silver sulfadiazine), elemental silver, antibiotics, bactericidal enzymes (such as lysozyme), wound healing agents (such as cytokines including but not limited to PDGF, TGF; thymosin), hyaluronic acid as a wound healing agent, wound sealants (such as fibrin with or without thrombin), cellular attractant and scaffolding reagents (such as added fibronectin) and the like. In a specific example, the collagen biofabric may be impregnated with at least one growth factor, for example, fibroblast growth factor, epithelial growth factor, etc. The biofabric may also be impregnated with small organic molecules such as specific inhibitors of particular biochemical processes e.g., membrane receptor inhibitors, kinase inhibitors, growth inhibitors, anticancer drugs, antibiotics, etc. Impregnating the collagen biofabric with a bioactive compound may be accomplished, e.g., by immersing the collagen biofabric in a solution of the bioactive compound of the desired concentration for a time sufficient to allow the collagen biofabric to absorb and to equilibrate with the solution.

In other embodiments, the collagen biofabric may be combined with a hydrogel to form a composite. Any hydrogel composition known to one skilled in the art is encompassed within the invention, e.g., any of the hydrogel compositions disclosed in the following reviews: Graham, 1998, Med. Device Technol. 9(1): 18-22; Peppas et al., 2000, Eur. J Pharm. Biopharm. 50(1): 27-46; Nguyen et al., 2002, Biomaterials, 23(22): 4307-14; Henincl et al., 2002, Adv. Drug Deliv. Rev 54(1): 13-36; Skelhorne et al., 2002, Med. Device. Technol. 13(9): 19-23; Schmedlen et al., 2002, Biomaterials 23: 4325-32; all of which are incorporated herein by reference in their entirety. In a specific embodiment, the hydrogel composition is applied on the collagen biofabric, i.e., disposed on the surface of the collagen biofabric. The hydrogel composition for example, may be sprayed onto the collagen biofabric or coated onto the surface of the collage biofabric, or the biofabric may be soaked, bathed or saturated with the hydrogel composition. In another specific embodiment, the hydrogel is sandwiched between two or more layers of collagen biofabric. In an even more specific embodiment, the hydrogel is sandwiched between two layers of collagen biofabric, wherein the edges of the two layers of biofabric are sealed so as to substantially or completely contain the hydrogel.

The hydrogels useful in the methods and compositions of the invention can be made from any water-interactive, or water soluble polymer known in the art, including but not limited to, polyvinylalcohol (PVA), polyhydroxyehthyl methacrylate, polyethylene glycol, polyvinyl pyrrolidone, hyaluronic acid, alginate, collagen, gelatin, dextran or derivatives and analogs thereof.

In some embodiments, the collagen biofabric of the invention comprises one or more bioactive compounds and is combined with a hydrogel. For example, the collagen biofabric can be impregnated with one or more bioactive compounds prior to being combined with a hydrogel. In other embodiments, the hydrogel composition is further impregnated with one or more bioactive compounds prior to, or after, being combined with a collagen biofabric of the invention, for example, the bioactive compounds described in Section 4.2.2, below.
4.2.2 Bioative Compounds

The collagen biofabric used in the methods of the invention may comprise (e.g., be impregnated with or coated with) one or more bioactive or medicinal compounds, such as small organic molecules (e.g., drugs), antibiotics, antiviral agents, antimicrobial agents, anti-inflammatory agents, antiproliferative agents, cytokines, enzyme or protein inhibitors, antihistamines, and the like. In various embodiments, the collagen biofabric may be coated or impregnated with antibiotics (such as Clindamycin, Minocycline, Doxycycline, Gentamycin), hormones, growth factors, anti-tumor agents, anti-flngal agents, anti-viral agents, pain medications (including XYLOCAINE®, Lidocaine, Procaine, Novocaine, etc.), antihistamines (e.g., diphenhydramine, BENADRYL®, etc.), anti-inflammatory agents, anti-infectives including but not limited to silver (such as silver salts, including but not limited to silver nitrate and silver sulfadiazine), elemental silver, antibiotics, bactericidal enzymes (such as lysozome), wound healing agents (such as cytokines including but not limited to PDGF (e.g., REGRANEX®), TGF; thymosin), hyaluronic acid as a wound healing agent, wound sealants (such as fibrin with or without thrombin), cellular attractant and scaffolding reagents (such as fibronectin), and the like, or combinations of any of the foregoing, or of the foregoing and other compounds not listed. Such impregnation or coating may be accomplished by any means known in the art, and a portion or the whole of the collagen biofabric may be so coated or impregnated.

The collagen biofabric, or composites comprising collagen biofabric, may comprise any of the compounds listed herein, without limitation, individually or in any combination. Any of the biologically active compounds listed herein, and others useful in the context of the sclera or eye, may be formulated by known methods for immediate release or extended release. Additionally, the collagen biofabric may comprise two or more biologically active compounds in different manners; e.g., the biofabric may be impregnated with one biologically active compound and coated with another. In another embodiment, the collagen biofabric comprises one biologically active compound formulated for extended release, and a second biologically active compound formulated for immediate release.

Wound healing, including the healing of tympanic membranes, including perforated tympanic membranes, requires adequate nutrition, particularly the presence of iron, zinc, vitamin C, arginine, and the like. Thus, the collagen biofabric may be impregnated or coated with a physiologically-available form of one or more nutrients required for wound healing. Preferably, the nutrient is formulated for extended release.

The collagen biofabric, or composite comprising collagen biofabric, may comprise an antibiotic. In certain embodiments, the antibiotic is a macrolide (e.g., tobramycin (TOBI®)), a cephalosporin (e.g., cephalexin (KEFLEX®)), cephradine (VELOSEF®)), cefuroxime (CEFTIN®, cefprozil (CEFZIL®), cefaclor (CECLOR®), cefixime (SUPRAX® or cefadroxil (DURICEF®), a clarithromycin (e.g., clarithromycin (Biaxin)), an erythromycin (e.g., erythromycin (EMYCIN®)), a penicillin (e.g., penicillin V (V-CILINK® or PEN VEEK®)) or a quinolone (e.g., ofloxacin (FLOXIN®), ciprofloxacin (CIPRO®) omorfloxacin (NOROXIN®)), aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefinetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), macrolides (e.g., azithromycin, carbomycin, clarithomycin, dirithromycin, erythromycin, and erythromycin acistrate), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, ciprofloxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin and tuberin.

In certain embodiments, the collagen biofabric may be coated or impregnated with an antifungal agent. Suitable antifungal agents include but are not limited to amphotericin B, itraconazole, ketoconazole, fluconazole, intrathecal, flucytosine, miconazole, butoconazole, clotrimazole, nystatin, terconazole, tioconazole, ciclopirox, econazole, haloprogrin, naftifine, terbinafine, undecylenate, and griseofuldin.

In certain other embodiments, the collagen biofabric, or a composite comprising collagen biofabric, is coated or impregnated with an anti-inflammatory agent. Useful anti-inflammatory agents include, but are not limited to, non-steroidal anti-inflammatory drugs such as salicylic acid, acetylsalicylic acid, methyl salicylate, diflunisal, salsalate, olsalazine, sulfasalazine, acetaminophen, indomethacin, sulindac, etodolac, mefenamic acid, meclofenamate sodium, tolmetin, ketorolac, dichlofenac, ibuprofen, naproxen, naproxen sodium, fenoprofen, ketoprofen, flurbinprofen, oxaprozin, piroxicam, meloxicam, ampiroxicam, droxicam, pivoxicam, tenoxicam, nabumetome, phenylbutazone, oxyphenbutazone, antipyrine, aminopyrine, apazone and nimesulide; leukotriene antagonists including, but not limited to, zileuton, aurothioglucose, gold sodium thiomalate and auranofin; and other anti-inflammatory agents including, but not limited to, methotrexate, colchicine, allopurinol, probenecid, sulfinpyrazone and benzbromarone.

In certain embodiments, the collagen biofabric, or a composite comprising collagen biofabric, is coated or impregnated with an antiviral agent. Useful antiviral agents include, but are not limited to, nucleoside analogs, such as zidovudine, acyclovir, gangcyclovir, vidarabine, idoxuridine, trifluridine, and ribavirin, as well as foscarnet, amantadine, rimantadine, saquinavir, indinavir, ritonavir, and the alpha-interferons.

The collagen biofabric, or a composite comprising collagen biofabric, may also be coated or impregnated with a cytokine receptor modulator. Examples of cytokine receptor modulators include, but are not limited to, soluble cytokine receptors (e.g., the extracellular domain of a TNF-α receptor or a fragment thereof, the extracellular domain of an IL-1 0 receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8,IL-9, IL-10, IL-11, IL-12, IL-15, TNF-α, TNF-β, interferon (IFN)-α, IFN-β, IFN-γ, and GM-CSF), anti-cytokine receptor antibodies (e.g., anti-IFN receptor antibodies, anti-IL-2 receptor antibodies (e.g., Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (e. g., anti-IFN antibodies, anti-TNF-α antibodies, anti-IL-10 antibodies, anti-IL-6 antibodies, anti-IL-8 antibodies (e.g., ABX-IL-8 (Abgenix)), and anti-IL-12 antibodies). In a specific embodiment, a cytokine receptor modulator is IL-4, IL-10, or a fragment thereof. In another embodiment, a cytokine receptor modulator is an anti-IL-1 antibody, anti-IL-6 antibody, anti-IL-12 receptor antibody, or anti-TNF-α antibody. In another embodiment, a cytokine receptor modulator is the extracellular domain of a TNF-α receptor or a fragment thereof. In certain embodiments, a cytokine receptor modulator is not a TNF-α antagonist.

In a preferred embodiment, proteins, polypeptides or peptides (including antibodies) that are utilized as immunomodulatory agents are derived from the same species as the recipient of the proteins, polypeptides or peptides so as to reduce the likelihood of an immune response to those proteins, polypeptides or peptides. In another preferred embodiment, when the subject is a human, the proteins, polypeptides, or peptides that are utilized as immunomodulatory agents are human or humanized.

The collagen biofabric, or a composite comprising collagen biofabric, may also be coated or impregnated with a cytokine. Examples of cytokines include, but are not limited to, colony stimulating factor 1 (CSF-1), interleukin-2 (IL-2), interleukin-3 (IL-3), interleukin-4 (IL-4), interleukin-5 (IL-5), interleukin-6 (IL-6), interleukin-7 (IL-7), interleukin-9 (IL-9), interleukin-10 (IL-10), interleukin-12 (IL-12), interleukin 15 (IL-15), interleukin 18 (IL-18), insulin-like growth factor 1 (IGF-1), platelet derived growth factor (PDGF), erythropoietin (Epo), epidermal growth factor (EGF), fibroblast growth factor (FGF) (basic or acidic), granulocyte macrophage stimulating factor (GM-CSF), granulocyte colony stimulating factor (G-CSF), heparin binding epidermal growth factor (HEGF), macrophage colony stimulating factor (M-CSF), prolactin, and interferon (IFN), e.g., IFN-alpha, and IFN-gamma), transforming growth factor alpha (TGF-α), TGFβ1, TGFβ2, tumor necrosis factor alpha (TNF-α), vascular endothelial growth factor (VEGF), hepatocyte growth factor (HGF), etc.

The collagen biofabric may also be coated or impregnated with a hormone. Examples of hormones include, but are not limited to, luteinizing hormone releasing hormone (LHRH), growth hormone (GH), growth hormone releasing hormone, ACTH, somatostatin, somatotropin, somatomedin, parathyroid hormone, hypothalamic releasing factors, insulin, glucagon, enkephalins, vasopressin, calcitonin, heparin, low molecular weight heparins, heparinoids, synthetic and natural opioids, insulin thyroid stimulating hormones, and endorphins. Examples of β-interferons include, but are not limited to, interferon β1-a and interferon β1-b.

The collagen biofabric, or composite comprising collagen biofabric, may also be coated or impregnated with an alkylating agent. Examples of alkylating agents include, but are not limited to nitrogen mustards, ethylenimines, methylmelamines, alkyl sulfonates, nitrosoureas, triazenes, mechlorethamine, cyclophosphamide, ifosfamide, melphalan, chlorambucil, hexamethylmelaine, thiotepa, busulfan, carmustine, streptozocin, dacarbazine and temozolomide.

The collagen biofabric, or a composite comprising collagen biofabric, may also be coated or impregnated with an immunomodulatory agent, including but not limited to methothrexate, leflunomide, cyclophosphamide, cyclosporine A, macrolide antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), T cell receptor modulators, and cytokine receptor modulators. peptide mimetics, and antibodies (e.g., human, humanized, chimeric, monoclonal, polyclonal, Fvs, ScFvs, Fab or F(ab)₂ fragments or epitope binding fragments), nucleic acid molecules (e.g., antisense nucleic acid molecules and triple helices), small molecules, organic compounds, and inorganic compounds. In particular, immunomodulatory agents include, but are not limited to, methothrexate, leflunomide, cyclophosphamide, cytoxan, Immuran, cyclosporine A, minocycline, azathioprine, antibiotics(e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), T cell receptor modulators, and cytokine receptor modulators. Examples of T cell receptor modulators include, but are not limited to, anti-T cell receptor antibodies (e.g., anti-CD4 antibodies (e.g., cM-T412 (Boeringer), IDEC-CE9.Is (IDEC and SKB), mAB 4162W94, Orthoclone and OKTcdr4a (Janssen-Cilag)), anti-CD3 antibodies (e.g., Nuvion (Product Design Labs), OKT3 (Johnson & Johnson), or Rituxan (IDEC)), anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies (e.g., IDEC-131(IDEC)), anti-CD52 antibodies (e.g., CAMPATH 1H (Ilex)), anti-CD2 antibodies, anti-CD11a antibodies (e.g., Xanelim (Genentech)), and anti-B7 antibodies (e.g., IDEC-114) (IDEC))) and CTLA4-immunoglobulin. In a specific embodiment, a T cell receptor modulator is a CD2 antagonist. In other embodiments, a T cell receptor modulator is not a CD2 antagonist. In another specific embodiment, a T cell receptor modulator is a CD2 binding molecule, preferably MEDI-507. In other embodiments, a T cell receptor modulator is not a CD2 binding molecule.

The collagen biofabric, or composite comprising collagen biofabric, may also be coated or impregnated with a class of immunomodulatory compounds known as IMIDs®. As used herein and unless otherwise indicated, the term “IMiD” and “IMIDs®” (Celgene Corporation) encompasses small organic molecules that markedly inhibit TNF-α, LPS induced monocyte IL1βB and IL12, and partially inhibit IL6 production. Specific immunomodulatory compounds are discussed below.

Specific examples of such immunomodulatory compounds, include, but are not limited to, cyano and carboxy derivatives of substituted styrenes such as those disclosed in U.S. Pat. No. 5,929,117; 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. Nos. 5,874,448 and 5,955,476; the tetra substituted 2-(2,6-dioxopiperdin-3-yl)-1-oxoisoindolines described in U.S. Pat. No. 5,798,368; 1-oxo and 1,3-dioxo-2-(2,6-dioxopiperidin-3-yl) isoindolines (e.g., 4-methyl derivatives of thalidomide), including, but not limited to, those disclosed in U.S. Pat. Nos. 5,635,517, 6,476,052, 6,555,554, and 6,403,613; 1-oxo and 1,3-dioxoisoindolines substituted in the 4- or 5-position of the indoline ring (e.g., 4-(4-amino-1,3-dioxoisoindoline-2-yl)-4-carbamoylbutanoic acid) described in U.S. Pat. No. 6,380,239; isoindoline-1-one and isoindoline-1,3-dione substituted in the 2-position with 2,6-dioxo-3-hydroxypiperidin-5-yl (e.g., 2-(2,6-dioxo-3-hydroxy-5-fluoropiperidin-5-yl)-4-aminoisoindolin-1-one) described in U.S. Pat. No. 6,458,810; a class of non-polypeptide cyclic amides disclosed in U.S. Pat. Nos. 5,698,579 and 5,877,200; aminothalidomide, as well as analogs, hydrolysis products, metabolites, derivatives and precursors of aminothalidomide, and substituted 2-(2,6-dioxopiperidin-3-yl) phthalimides and substituted 2-(2,6-dioxopiperidin-3-yl)-1-oxoisoindoles such as those described in U.S. Pat. Nos. 6,281,230 and 6,316,471; and isoindole-imide compounds such as those described in U.S. patent application Ser. No. 09/972,487 filed on Oct. 5, 2001, U.S. patent application Ser. No. 10/032,286 filed on Dec. 21, 2001, and International Application No. PCT/US01/50401 (International Publication No. WO 02/059106). The entireties of each of the patents and patent applications identified herein are incorporated herein by reference. Immunomodulatory compounds do not include thalidomide.

Other specific immunomodulatory compounds include, but are not limited to, 1-oxo- and 1,3 dioxo-2-(2,6-dioxopiperidin-3-yl) isoindolines substituted with amino in the benzo ring as described in U.S. Pat. No. 5,635,517 which is incorporated herein by reference. These compounds have the structure I: 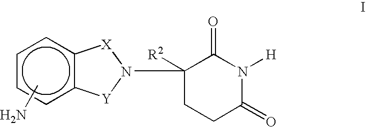
in which one of X and Y is C═O, the other of X and Y is C═O or CH₂, and R² is hydrogen or lower alkyl, in particular methyl. Specific immunomodulatory compounds include, but are not limited to:
1-oxo-2-(2,6-dioxopiperidin-3-yl)-4-aminoisoindoline;
1-oxo-2-(2,6-dioxopiperidin-3-yl)-5-aminoisoindoline;
1-oxo-2-(2,6-dioxopiperidin-3-yl)-6-aminoisoindoline;
1-oxo-2-(2,6-dioxopiperidin-3-yl)-7-aminoisoindoline;
1,3-dioxo-2-(2,6-dioxopiperidin-3-yl)-4-aminoisoindoline; and
1,3-dioxo-2-(2,6-dioxopiperidin-3-yl)-5-aminoisoindoline.

Other specific immunomodulatory compounds belong to a class of substituted 2-(2,6-dioxopiperidin-3-yl) phthalimides and substituted 2-(2,6-dioxopiperidin-3-yl)-1-oxoisoindoles, such as those described in U.S. Pat. Nos. 6,281,230; 6,316,471; 6,335,349; and 6,476,052, and International Patent Application No. PCT/US97/13375 (International Publication No. WO 98/03502), each of which is incorporated herein by reference. Representative compounds are of formula:

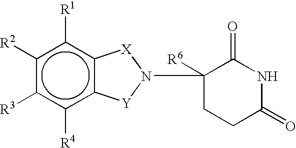

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzyl, or halo;

provided that R⁶ is other than hydrogen if X and Y are C═O and (i) each of R¹, R², R³, and R⁴ is fluoro or (ii) one of R¹, R², R³, or R⁴ is amino.

Compounds representative of this class are of the formulas: 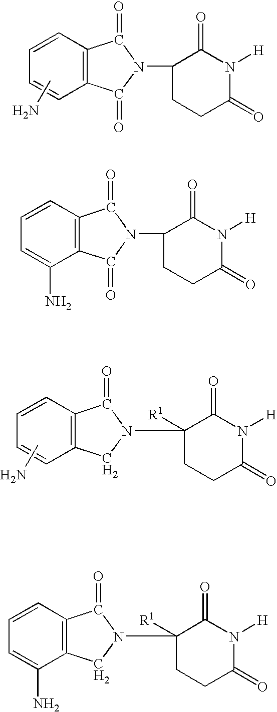
wherein R¹ is hydrogen or methyl. In a separate embodiment, the invention encompasses the use of enantiomerically pure forms (e.g. optically pure (R) or (S) enantiomers) of these compounds.

Still other specific immunomodulatory compounds belong to a class of isoindole-imides imides disclosed in U.S. Patent Application Publication Nos. US 2003/0096841 and US 2003/0045552, and Interational Application No. PCT/US01/50401 (International Publication No. WO 02/059106), each of which are incorporated herein by reference. Representative compounds are of formula II: 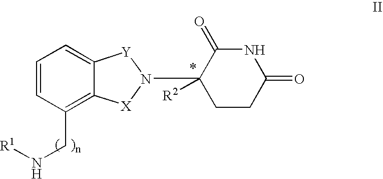
and pharmaceutically acceptable salts, hydrates, solvates, claturates, enantiomers, diastereomers, racemates, and mixtures of stereoisomers thereof, wherein:

one of X and Y is C═O and the other is CH₂ or C═O;

R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′}or (C₁-C₈)alkyl-O(CO)R⁵;

R² is H, F, benzyl, (C₁-C₈)alkyl, (C₂-C₈)alkenyl, or (C₂-C₈)alkynyl;

R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;

R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;

R⁵ is (C₁-C₈)alkyl, (C₂-C₈)alkenyl, (C₂-C₈)alkynyl, benzyl, aryl, or (C₂-C₅)heteroaryl;

each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O-R⁵ or the R⁶ groups can join to form a heterocycloalkyl group;

n is 0 or 1; and

* represents a chiral-carbon center.

In specific compounds of formula II, when n is 0 then R¹ is (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(O)OR⁴, (C₁-C₈)alkyl-N(R⁶)₂, (C₁-C₈)alkyl-OR⁵, (C₁-C₈)alkyl-C(O)OR⁵, C(S)NHR³, or (C₁-C₈)alkyl-O(CO)R⁵;

R² is H or (C₁-C₈)alkyl; and

R³ is (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₅-C₈)alkyl-N(R⁶)₂; (C₀-C₈)alkyl-NH—C(O)O—R⁵; (C₁-C₈)alkyl-OR⁵, (C₁-C₈)alkyl-C(O)OR⁵, (C₁-C₈)alkyl-O(CO)R⁵, or C(O)OR⁵; and the other variables have the same definitions.

In other specific compounds of formula II, R² is H or (C₁-C₄)alkyl.

In other specific compounds of formula II, R¹ is (C₁-C₈)alkyl or benzyl.

In other specific compounds of formula II, R¹ is H, (C₁-C₈)alkyl, benzyl, CH₂OCH₃, CH₂CH₂OCH₃, or

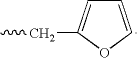

In another embodiment of the compounds of formula II, R¹ is 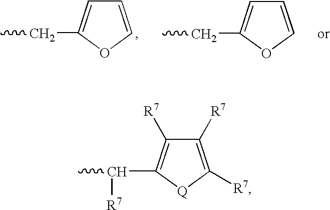
wherein Q is O or S, and each occurrence of R⁷ is independently H,(C₁-C₈)alkyl, (C₃-C₇)cycloalkyl, (C₂-C₈)alkenyl, (C₂-C₈)alkynyl, benzyl, aryl, halogen, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵, or adjacent occurrences of R⁷ can be taken together to form a bicyclic alkyl or aryl ring.

In other specific compounds of formula II, R¹ is C(O)R³.

In other specific compounds of formula II, R³ is (C₀-C₄)alkyl-(C₂-C₅)heteroaryl, (C₁-C₈)alkyl, aryl, or (C₀-C₄)alkyl-OR⁵.

In other specific compounds of formula II, heteroaryl is pyridyl, furyl, or thienyl.

In other specific compounds of formula II, R¹ is C(O)OR⁴.

In other specific compounds of formula II, the H of C(O)NHC(O) can be replaced with (C₁-C₄)alkyl, aryl, or benzyl.

Further examples of the compounds in this class include, but are not limited to: [2-(2,6-dioxo-piperidin-3-yl)-1,3-dioxo-2,3-dihydro-1H-isoindol-4-ylmethyl]-amide; (2-(2,6-dioxo-piperidin-3-yl)-1,3-dioxo-2,3-dihydro-1H-isoindol-4-ylmethyl)-carbamic acid tert-butyl ester; 4-(aminomethyl)-2-(2,6-dioxo(3-piperidyl))-isoindoline-1,3-dione; N-(2-(2,6-dioxo-piperidin-3-yl)-1,3-dioxo-2,3-dihydro-1H-isoindol-4-ylmethyl)-acetamide; N-{(2-(2,6-dioxo(3-piperidyl)-1,3-dioxoisoindolin-4-yl)methyl}cyclopropyl-carboxamide; 2-chloro-N-{(2-(2,6-dioxo(3-piperidyl))-1,3-dioxoisoindolin-4-yl)methyl}acetamide; N-(2-(2,6-dioxo(3-piperidyl))-1,3-dioxoisoindolin-4-yl)-3-pyridylcarboxamide; 3-{1-oxo-4-(benzylamino)isoindolin-2-yl}piperidine-2,6-dione; 2-(2,6-dioxo(3-piperidyl))-4-(benzylamino)isoindoline-1,3-dione; N-{(2-(2,6-dioxo(3-piperidyl))-1,3-dioxoisoindolin-4-yl)methyl}propanamide; N-{(2-(2,6-dioxo(3-piperidyl))-1,3-dioxoisoindolin-4-yl)methyl}-3-pyridylcarboxamide; N-{(2-(2,6-dioxo(3-piperidyl))-1,3-dioxoisoindolin-4-yl)methyl}heptanamide; N-{(2-(2,6-dioxo(3-piperidyl))-1,3-dioxoisoindolin-4-yl)methyl}-2-furylcarboxamide; {N-(2-(2,6-dioxo(3-piperidyl))-1,3-dioxoisoindolin-4-yl)carbamoyl}methyl acetate; N-(2-(2,6-dioxo(3-piperidyl))-1,3-dioxoisoindolin-4-yl)pentanamide; N-(2-(2,6-dioxo(3-piperidyl))-1,3-dioxoisoindolin-4-yl)-2-thienylcarboxamide; N-{[2-(2,6-dioxo(3-piperidyl))-1,3-dioxoisoindolin-4-yl]methyl}(butylamino)carboxamide; N-{[2-(2,6-dioxo(3-piperidyl))-1,3-dioxoisoindolin-4-yl]methyl}(octylamino)carboxamide; and N-{[2-(2,6dioxo(3-piperidyl))-1,3-dioxoisoindolin-4-yl]methyl}(benzylamino)carboxamide.

Still other specific immunomodulatory compounds belong to a class of isoindole-imides disclosed in U.S. Patent Application Publication Nos. US 2002/0045643, International Publication No. WO 98/54170, and U.S. Pat. No. 6,395,754, each of which is incorporated herein by reference. Representative compounds are of formula III: 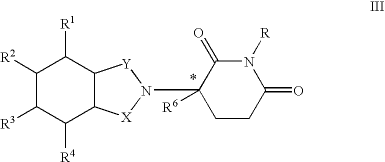
and pharmaceutically acceptable salts, hydrates, solvates, clathrates, enantiomers, diastereomers, racemates, and mixtures of stereoisomers thereof, wherein:

one of X and Y is C═O and the other is CH₂ or C═O;

R is H or CH₂OCOR′;

(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;

R⁵ is hydrogen or alkyl of 1 to 8 carbons

R⁶ hydrogen, alkyl of 1 to 8 carbon atoms, benzo, chloro, or fluoro;

R′ is R⁷—CHR¹⁰—N(R⁸R⁹);

R⁷ is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;

R¹⁰ is hydrogen, alkyl of to 8 carbon atoms, or phenyl; and

* represents a chiral-carbon center.

Other representative compounds are of formula:

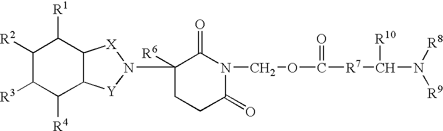

wherein:

one of X and Y is C═O and the other of X and Y is C═O or CH₂;

(i) each of R¹, R², R³, or R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵ is hydrogen or alkyl of 1 to 8 carbon atoms;

R⁶ is hydrogen, alkyl of 1 to 8 carbon atoms, benzo, chloro, or fluoro;

R⁷is m-phenylene or p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂ X¹CH₂CH₂— in which X¹ is —O—, —S—, or —NH—;

R¹⁰ is hydrogen, alkyl of to 8 carbon atoms, or phenyl.

Other representative compounds are of formula:

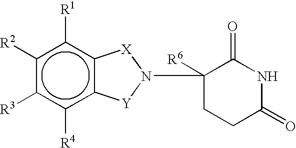

in which:

one of X and Y is C═O and the other of X and Y is C═O or CH₂;

each of R¹, R², R³, and R⁴, independently of the others, is halo, alkyl of 1 to 4 carbon atoms, or alkoxy of 1 to 4 carbon atoms or (ii) one of R¹, R², R³, and R⁴ is nitro or protected amino and the remaining of R¹, R², R³, and R⁴ are hydrogen; and

R⁶ is hydrogen, alkyl of 1 to 8 carbon atoms, benzo, chloro, or fluoro.

Other representative compounds are of formula:

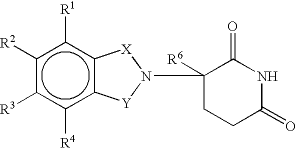

in which:

one of X and Y is C═O and the other of X and Y is C═O or CH₂;

(i) each of R¹, R², R³, and R⁴, independently of the others, is halo, alkyl of 1 to 4 carbon atoms, or alkoxy of 1 to 4 carbon atoms or (ii) one of R¹, R², R³, and R⁴ is —NHR⁵ and the remaining of R¹, R², R³, and R⁴ are hydrogen;

R⁵is hydrogen, alkyl of 1 to 8 carbon atoms, or CO—R⁷—CH(R¹⁰)NR⁸R⁹ in which each of R⁷, R⁸, R⁹, and R¹⁰ is as herein defined; and

R⁶ is alkyl of 1 to 8 carbon atoms, benzo, chloro, or fluoro.

Specific examples of the compounds are of formula:

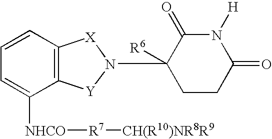

in which:

one of X and Y is C═O and the other of X and Y is C═O or CH₂;

R⁶ is hydrogen, alkyl of 1 to 8 carbon atoms, benzyl, chloro, or fluoro;

R⁷is m-phenylene, p-phenylene or —(CₙH₂ₙ)— in which n has a value of 0 to 4;

each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X¹CH₂CH₂— in which X¹ is —O—, —S— or —NH—; and

R¹⁰ is hydrogen, alkyl of 1 to 8 carbon atoms, or phenyl.

Preferred immunomodulatory compounds are 4-(amino)-2-(2,6-dioxo(3-piperidyl))-isoindoline-1,3-dione and 3-(4-amino-1-oxo-1,3-dihydro-isoindol-2-yl)-piperidine-2,6-dione. The compounds can be obtained via standard, synthetic methods (see e.g., U.S. Pat. No. 5,635,517, incorporated herein by reference). The compounds are available from Celgene Corporation, Warren, N.J. 4-(Amino)-2-(2,6-dioxo(3-piperidyl))-isoindoline-1,3-dione has the following chemical structure:

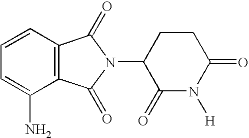

The compound 3-(4-amino-1-oxo-1,3-dihydro-isoindol-2-yl)-piperidine-2,6-dione has the following chemical structure:

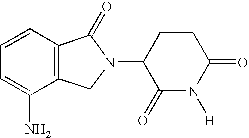

In another embodiment, specific immunomodulatory compounds encompass polymorphic forms of 3-(4-amino-1-oxo-1,3 dihydro-isoindol-2-yl)-piperidene-2,6-dione such as Form A, B, C, D, E, F, G and H, disclosed in U.S. provisional application No. 60/499,723 filed on Sep. 4, 2003, and U.S. non-provisional application No. 10/934,863, filed Sep. 3, 2004, both of which are incorporated herein by reference. For example, Form A of 3-(4-amino-1-oxo-1,3 dihydro-isoindol-2-yl)-piperidene-2,6-dione is an unsolvated, crystalline material that can be obtained from non-aqueous solvent systems. Form A has an X-ray powder diffraction pattern comprising significant peaks at approximately 8, 14.5, 16, 17.5, 20.5, 24 and 26 degrees 2θ, and has a differential scanning calorimetry melting temperature maximum of about 270° C. Form A is weakly or not hygroscopic and appears to be the most thermodynamically stable anhydrous polymorph of 3-(4-amino-1-oxo-1,3 dihydro-isoindol-2-yl)-piperidine-2,6-dione discovered thus far.

Form B of 3-(4-amino-1-oxo-1,3 dihydro-isoindol-2-yl)-piperidene-2,6-dione is a hemihydrated, crystalline material that can be obtained from various solvent systems, including, but not limited to, hexane, toluene, and water. Form B has an X-ray powder diffraction pattern comprising significant peaks at approximately 16, 18, 22 and 27 degrees 2θ, and has endotherms from DSC curve of about 146 and 268° C., which are identified dehydration and melting by hot stage microscopy experiments. Interconversion studies show that Form B converts to Form E in aqueous solvent systems, and converts to other forms in acetone and other anhydrous systems.

Form C of 3-(4-amino-1-oxo-1,3 dihydro-isoindol-2-yl)-piperidene-2,6-dione is a hemisolvated crystalline material that can be obtained from solvents such as, but not limited to, acetone. Form C has an X-ray powder diffraction pattern comprising significant peaks at approximately 15.5 and 25 degrees 2θ, and has a differential scanning calorimetry melting temperature maximum of about 269° C. Form C is not hygroscopic below about 85% RH, but can convert to Form B at higher relative humidities.

Form D of 3-(4-amino-1-oxo-1,3 dihydro-isoindol-2-yl)-piperidene-2,6-dione is a crystalline, solvated polymorph prepared from a mixture of acetonitrile and water. Form D has an X-ray powder diffraction pattern comprising significant peaks at approximately 27 and 28 degrees 2θ, and has a differential scanning calorimetry melting temperature maximum of about 270° C. Form D is either weakly or not hygroscopic, but will typically convert to Form B when stressed at higher relative humidities.

Form E of 3-(4-amino-1-oxo-1,3 dihydro-isoindol-2-yl)-piperidene-2,6-dione is a dihydrated, crystalline material that can be obtained by slurrying 3-(4-amino-1-oxo-1,3 dihydro-isoindol-2-yl)-piperidene-2,6-dione in water and by a slow evaporation of 3-(4-amino-1-oxo-1,3 dihydro-isoindol-2-yl)-piperidene-2,6-dione in a solvent system with a ratio of about 9:1 acetone:water. Form E has an X-ray powder diffraction pattern comprising significant peaks at approximately 20, 24.5 and 29 degrees 2θ, and has a differential scanning calorimetry melting temperature maximum of about 269° C. Form E can convert to Form C in an acetone solvent system and to Form G in a THF solvent system. In aqueous solvent systems, Form E appears to be the most stable form. Desolvation experiments performed on Form E show that upon heating at about 125° C. for about five minutes, Form E can convert to Form B. Upon heating at 175° C. for about five minutes, Form B can convert to Form F.

Form F of 3-(4-amino-1-oxo-1,3 dihydro-isoindol-2-yl)-piperidene-2,6-dione is an unsolvated, crystalline material that can be obtained from the dehydration of Form E. Form F has an X-ray powder diffraction pattern comprising significant peaks at approximately 19, 19.5 and 25 degrees 2θ, and has a differential scanning calorimetry melting temperature maximum of about 269° C.

Form G of 3-(4-amino-1-oxo-1,3 dihydro-isoindol-2-yl)-piperidene-2,6-dione is an unsolvated, crystalline material that can be obtained from slurrying forms B and E in a solvent such as, but not limited to, tetrahydrofuran (THF). Form G has an X-ray powder diffraction pattern comprising significant peaks at approximately 21, 23 and 24.5 degrees 2θ, and has a differential scanning calorimetry melting temperature maximum of about 267° C.

Form H of 3-(4-amino-1-oxo-1,3 dihydro-isoindol-2-yl)-piperidene-2,6-dione is a partially hydrated (about 0.25 moles) crystalline material that can be obtained by exposing Form E to 0% relative humidity. Form H has an X-ray powder diffraction pattern comprising significant peaks at approximately 15, 26 and 31 degrees 2θ, and has a differential scanning calorimetry melting temperature maximum of about 269° C.

Other specific immunomodulatory compounds include, but are not limited to, 1-oxo-2-(2,6-dioxo-3-fluoropiperidin-3yl) isoindolines and 1,3-dioxo-2-(2,6-dioxo-3-fluoropiperidine-3-yl) isoindolines such as those described in U.S. Pat. Nos. 5,874,448 and 5,955,476, each of which is incorporated herein by reference. Representative compounds are of formula:

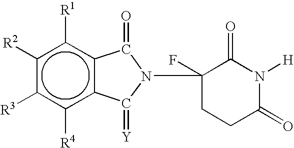

wherein Y is oxygen or H and

each of R¹, R², R³, and R⁴, independently of the others, is hydrogen, halo, alkyl of 1 to 4 carbon atoms, alkoxy of 1 to 4 carbon atoms, or amino.

Other specific immunomodulatory compounds include, but are not limited to, the tetra substituted 2-(2,6-dioxopiperdin-3-yl)-1-oxoisoindolines described in U.S. Pat. No. 5,798,368, which is incorporated herein by reference. Representative compounds are of formula:

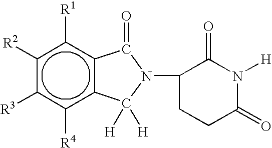

wherein each of R¹, R², R³, and R⁴, independently of the others, is halo, alkyl of 1 to 4 carbon atoms, or alkoxy of 1 to 4 carbon atoms.

Other specific immunomodulatory compounds include, but are not limited to, 1-oxo and 1,3-dioxo-2-(2,6-dioxopiperidin-3-yl) isoindolines disclosed in U.S. Pat. No. 6,403,613, which is incorporated herein by reference. Representative compounds are of formula:

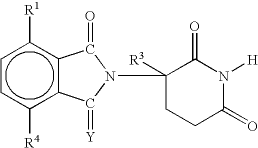

in which

Y is oxygen or H₂,

a first of R¹ and R² is halo, alkyl, alkoxy, alkylamino, dialkylamino, cyano, or carbamoyl, the second of R¹ and R², independently of the first, is hydrogen, halo, alkyl, alkoxy, alkylamino, dialkylamino, cyano, or carbamoyl, and

R³ is hydrogen, alkyl, or benzyl.

Specific examples of the compounds are of formula:

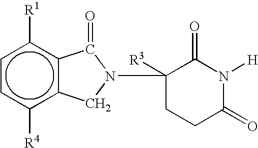

wherein a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl,

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl, and

R³ is hydrogen, alkyl of from 1 to 4 carbon atoms, or benzyl. Specific examples include, but are not limited to, 1-oxo-2-(2,6-dioxopiperidin-3-yl)-4-methylisoindoline.

Other representative compounds are of formula:

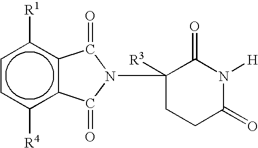

wherein a first of R¹ and R² is halo, alkyl of from 1 to 4 carbon atoms, alkoxy of from 1 to 4 carbon atoms, dialkylamino in which each alkyl is of from 1 to 4 carbon atoms, cyano, or carbamoyl,

the second of R¹ and R², independently of the first, is hydrogen, halo, alkyl of from 1 to 4 carbon atoms, alkoxy of from 1 to 4 carbon atoms, alkylamino in which alkyl is of from 1 to 4 carbon atoms, dialkylamino in which each alkyl is of from 1 to 4 carbon atoms, cyano, or carbamoyl, and

R³ is hydrogen, alkyl of from 1 to 4 carbon atoms, or benzyl.

Specific examples include, but are not limited to, 1-oxo-2-(2,6-dioxopiperidin-3-yl)-4-methylisoindoline.

Other specific immunomodulatory compounds include, but are not limited to, 1-oxo and 1,3-dioxoisoindolines substituted in the 4- or 5-position of the indoline ring described in U.S. Pat. No. 6,380,239 and co-pending U.S. application Ser. No. 10/900,270, filed Jul. 28, 2004, which are incorporated herein by reference. Representative compounds are of formula:

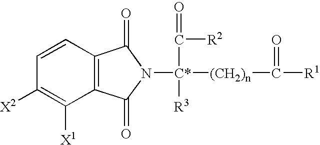

in which the carbon atom designated C* constitutes a center of chirality (when n is not zero and R¹ is not the same as R²); one of X¹ and X² is amino, nitro, alkyl of one to six carbons, or NH-Z, and the other of X¹ or X² is hydrogen; each of R¹ and R² independent of the other, is hydroxy or NH-Z; R³ is hydrogen, alkyl of one to six carbons, halo, or haloalkyl; Z is hydrogen, aryl, alkyl of one to six carbons, formyl, or acyl of one to six carbons; and n has a value of 0, 1, or 2; provided that if X¹ is amino, and n is 1 or 2, then R¹ and R² are not both hydroxy; and the salts thereof.

Further representative compounds are of formula: 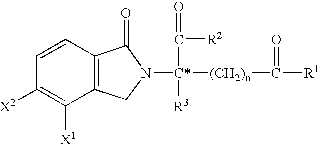
in which the carbon atom designated C* constitutes a center of chirality when n is not zero and R¹ is not R²; one of X¹ and X² is amino, nitro, alkyl of one to six carbons, or NH-Z, and the other of X¹ or X² is hydrogen; each of R¹ and R² independent of the other, is hydroxy or NH-Z; R³ is alkyl of one to six carbons, halo, or hydrogen; Z is hydrogen, aryl or an alkyl or acyl of one to six carbons; and n has a value of 0, 1, or 2.

Specific examples include, but are not limited to, 2-(4-amino-1-oxo-1,3-dihydro-isoindol-2-yl)-4-carbamoyl-butyric acid and 4-(4-amino-1-oxo-1,3-dihydro-isoindol-2-yl)-4-cabamoyl-butyric acid, which have the following structures, respectively, and pharmaceutically acceptable salts, solvates, prodrugs, and stereoisomers thereof:

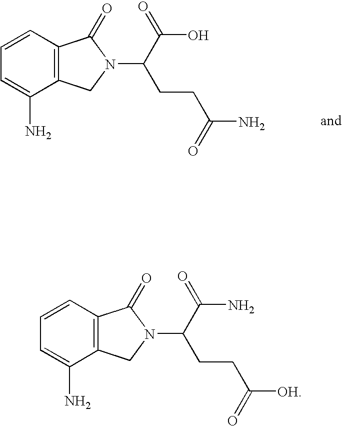

Other representative compounds are of formula: 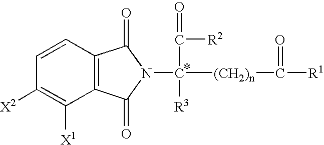
in which the carbon atom designated C* constitutes a center of chirality when n is not zero and R¹ is not R²; one of X¹ and X² is amino, nitro, alkyl of one to six carbons, or NH-Z, and the other of X¹ or X² is hydrogen; each of R¹ and R² independent of the other, is hydroxy or NH-Z; R³ is alkyl of one to six carbons, halo, or hydrogen; Z is hydrogen, aryl, or an alkyl or acyl of one to six carbons; and n has a value of 0, 1, or 2; and the salts thereof.

Specific examples include, but are not limited to, 4-carbamoyl-4-{4-[(furan-2-yl-methyl)-amino]-1,3-dioxo-1,3-dihydro-isoindol-2-yl}-butyric acid, 4-carbamoyl-2-{4-[(furan-2-yl-methyl)-amino]-1,3-dioxo-1,3-dihydro-isoindol-2-yl}-butyric acid, 2-{4-[(2-yl-methyl)-amino]-1,3-dioxo-1,3-dihydro-isoindol-2-yl}-4-phenylcarbamoyl-butyric acid, and 2-{4-[(furan-2-yl-methyl)-amino]-1,3-dioxo-1,3dihydro-isoindol-2-yl}-pentanedioic acid, which have the following structures, respectively, and pharmaceutically acceptable salts, solvate, prodrugs, and stereoisomers thereof:

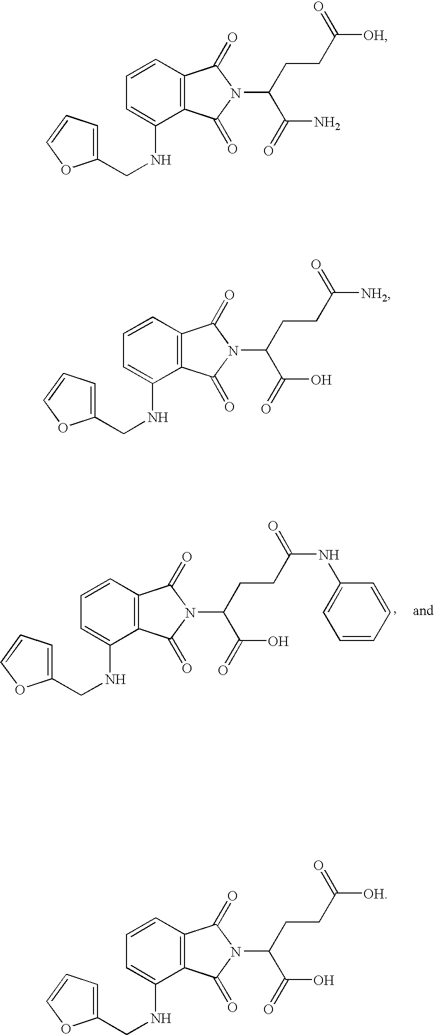

Other specific examples of the compounds are of formula:

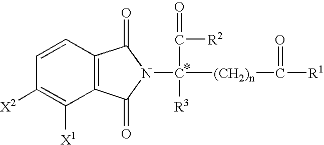

wherein one of X¹ and X² is nitro, or NH-Z, and the other of X¹ or X² is hydrogen;

each of R¹ and R², independent of the other, is hydroxy or NH-Z;

R³ is alkyl of one to six carbons, halo, or hydrogen;

Z is hydrogen, phenyl, an acyl of one to six carbons, or an alkyl of one to six carbons; and

n has a value of 0, 1, or 2;

provided that if one of X¹ and X² is nitro, and n is 1 or 2, then R¹ and R² are other than hydroxy; and

if —COR² and —(CH₂)ₙCOR¹ are different, the carbon atom designated C* constitutes a center of chirality. Other representative compounds are of formula:

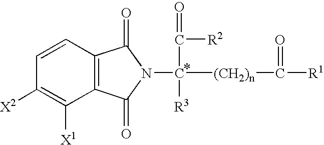

wherein one of X¹ and X² is alkyl of one to six carbons;

each of R¹ and R², independent of the other, is hydroxy or NH-Z;

R³ is alkyl of one to six carbons, halo, or hydrogen;

Z is hydrogen, phenyl, an acyl of one to six carbons, or an alkyl of one to six carbons; and

n has a value of 0, 1, or 2; and

if —COR² and —(CH₂)ₙCOR¹ are different, the carbon atom designated C* constitutes a center of chirality.

Still other specific immunomodulatory compounds include, but are not limited to, isoindoline-1-one and isoindoline-1,3-dione substituted in the 2-position with 2,6-dioxo-3-hydroxypiperidin-5-yl described in U.S. Pat. No. 6,458,810, which is incorporated herein by reference. Representative compounds are of formula:

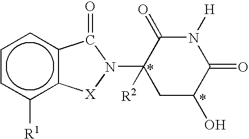

wherein:

the carbon atoms designated * constitute centers of chirality;

X is —C(O)— or —CH₂—;

R¹ is alkyl of 1 to 8 carbon atoms or —NHR³;

R² is hydrogen, alkyl of 1 to 8 carbon atoms, or halogen;

and

R³ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms,

benzyl, unsubstituted or substituted with alkyl of 1 to 8 carbon atoms, alkoxy of 1 to 8 carbon atoms, halo, amino, or alkylamino of 1 to 4 carbon atoms, or —COR⁴ in which

R⁴ is hydrogen,

alkyl of 1 to 8 carbon atoms, unsubstituted or substituted with alkoxy of 1 to 8 carbon atoms, halo, amino, or alkylamino of 1 to 4 carbon atoms,

cycloalkyl of 3 to 18 carbon atoms,

phenyl, unsubstituted or substituted with alkyl of 1 to 8 carbon atoms, alkoxy of 1 to 8 carbon atoms, halo, amino, or alkylamino of 1 to 4 carbon atoms, or

benzyl, unsubstituted or substituted with alkyl of 1 to 8 carbon atoms, alkoxy of 1 to 8 carbon atoms, halo, amino, or alkylamino of 1 to 4 carbon atoms.

The immunomodulatory compounds disclosed herein can either be commercially purchased or prepared according to the methods described in the patents or patent publications disclosed herein. Further, optically pure compounds can be asymmetrically synthesized or resolved using known resolving agents or chiral columns as well as other standard synthetic organic chemistry techniques.

As used herein and unless otherwise indicated, the term “pharmaceutically acceptable salt” encompasses non-toxic acid and base addition salts of the compound to which the term refers. Acceptable non-toxic acid addition salts include those derived from organic and inorganic acids or bases know in the art, which include, for example, hydrochloric acid, hydrobromic acid, phosphoric acid, sulfuric acid, methanesulphonic acid, acetic acid, tartaric acid, lactic acid, succinic acid, citric acid, malic acid, maleic acid, sorbic acid, aconitic acid, salicylic acid, phthalic acid, embolic acid, enanthic acid, and the like.

Compounds that are acidic in nature are capable of forming salts with various pharmaceutically acceptable bases. The bases that can be used to prepare pharmaceutically acceptable base addition salts of such acidic compounds are those that form non-toxic base addition salts, i.e., salts containing pharmacologically acceptable cations such as, but not limited to, alkali metal or alkaline earth metal salts and the calcium, magnesium, sodium or potassium salts in particular. Suitable organic bases include, but are not limited to, N,N-dibenzylethylenediamine, chloroprocaine, choline, diethanolamine, ethylenediamine, meglumaine (N-methylglucamine), lysine, and procaine.

As used herein, and unless otherwise specified, the term “solvate” means a compound of the present invention or a salt thereof, that further includes a stoichiometric or non-stoichiometric amount of solvent bound by non-covalent intermolecular forces. Where the solvent is water, the solvate is a hydrate.

As used herein and unless otherwise indicated, the term “prodrug” means a derivative of a compound that can hydrolyze, oxidize, or otherwise react under biological conditions (in vitro or in vivo) to provide the compound. Examples of prodrugs include, but are not limited to, derivatives of immunomodulatory compounds of the invention that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include derivatives of immunomodulatory compounds of the invention that comprise —NO, —NO₂, —ONO, or —ONO₂ moieties. Prodrugs can typically be prepared using well-known methods, such as those described in 1 Burger's Medicinal Chemistry and Drug Discovery, 172-178, 949-982 (Manfred E. Wolff ed., 5th ed. 1995), and Design of Prodrugs (H. Bundgaard ed., Elselvier, New York 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxymethyl, pivaloyloxymethyl, and pivaloyloxyethyl esters), lactonyl esters (such as phthalidyl and thiophthalidyl esters), lower alkoxyacyloxyalkyl esters (such as methoxycarbonyl-oxymethyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

As used herein, and unless otherwise specified, the term “stereoisomer” encompasses all enantiomerically/stereomerically pure and enantiomerically/stereomerically enriched compounds of this invention.

As used herein, and unless otherwise indicated, the term “stereomerically pure” or “enantiomerically pure” means that a compound comprises one stereoisomer and is substantially free of its counter stereoisomer or enantiomer. For example, a compound is stereomerically or enantiomerically pure when the compound contains 80%, 90%, or 95% or more of one stereoisomer and 20%, 10%, or 5% or less of the counter stereoisomer. In certain cases, a compound of the invention is considered optically active or stereomerically/enantiomerically pure (i.e., substantially the R-form or substantially the S-form) with respect to a chiral center when the compound is about 80% ee (enantiomeric excess) or greater, preferably, equal to or greater than 90% ee with respect to a particular chiral center, and more preferably 95% ee with respect to a particular chiral center.

As used herein, and unless otherwise indicated, the term “stereomerically enriched” or “enantiomerically enriched” encompasses racemic mixtures as well as other mixtures of stereoisomers of compounds of this invention (e.g., R/S=30/70, 35/65, 40/60, 45/55, 55/45, 60/40, 65/35 and 70/30). Various immunomodulatory compounds of the invention contain one or more chiral centers, and can exist as racemic mixtures of enantiomers or mixtures of diastereomers. This invention encompasses the use of stereomerically pure forms of such compounds, as well as the use of mixtures of those forms. For example, mixtures comprising equal or unequal amounts of the enantiomers of a particular immunomodulatory compounds of the invention may be used in methods and compositions of the invention. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind, 1972).

It should be noted that if there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

The amount of the bioactive compound coating or impregnating the collagen biofabric may vary, and will preferably depend upon the particular bioactive compound to be delivered, and the effect desired. For example, where the bioactive compound is an anti-inflammatory agent, the amount of the anti-inflammatory agent on or contained by the collagen biofabric is an amount sufficient to measurably reduce one or more symptoms or indicia of inflammation in the tympanic membrane, and/or area surrounding the tympanic membrane.

In various embodiments, the collagen biofabric of the invention may be coated with, or impregnated with, at least 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 100, 1250, 1500, 2000, 2500, 300, 3500, 4000, 4500, 5000, 5500, 6000, 6500, 7000, 7500, 8000, 8500, 9000, 9500, 10000, 20000, 30000, 40000, 50000, 60000, 70000, 80000, 90000, 100000, 200000, 300000, 400000, 500000, 600000, 700000, 800000, 900000 or at least 1000000 nanograms of a bioactive compound. In another embodiment, the collagen biofabric of the invention may be coated with, or impregnated with, no more than 0.1, 0.2, 0.3, 0.4, 0.5, 0.6, 0.7, 0.8, 0.9, 1, 2, 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 200, 300, 400, 500, 600, 700, 800, 900, 100, 1250, 1500, 2000, 2500, 300, 3500, 4000, 4500, 5000, 5500, 6000, 6500, 7000, 7500, 8000, 8500, 9000, 9500, 10000, 20000, 30000, 40000, 50000, 60000, 70000, 80000, 90000, 100000, 200000, 300000, 400000, 500000, 600000, 700000, 800000, 900000 or at least 1000000 nanograms of a bioactive compound.
4.2.3 Conformation of the Collagen Biofabric

The collagen biofabric may be formed into any shape or conformation that will facilitate its use in the methods of the invention. For example, the collagen biofabric can be formed into any shape or conformation that will facilitate the occlusion of a tympanic membrane perforation, particularly in the context of a tympanoplasty or myringoplasty. For example, the collagen biofabric may be provided in various sizes so as to enable an otolaryngologist, or other end user, to use or to cut an appropriately-sized piece for repair of a particular tympanic membrane. The collagen biofabric may, for example, be provided as square, rectangular, circular or oval shaped pieces, or may be cut to conform generally to the shape of a tympanic membrane. In various embodiments of the method, collagen biofabric pieces used to repair a tympanic membrane may be provided as pieces measuring approximately 1×1 cm, 1.5×1.5 cm, 2×2 cm, 2.5×2.5 cm, 3×3 cm, 3.5×3.5 cm, 4×4 cm, 4.5×4.5 cm, 5×5 cm, 1×1.5 cm, 1×2 cm, 1×2.5 cm, 1×3 cm, 1×3.5 cm, 1×4 cm, 1×4.5 cm, 1×5 cm, 1.5×2 cm, 1.5×2.5 cm, 1.5×3 cm, 1.5×3.5 cm, 1.5×4 cm, 1.5×4.5 cm , 2×2.5 cm, 2×3 cm, 2×3.5 cm, 2×4 cm, 2×4.5 cm, 2×5 cm, 2.5×3 cm, 2.5×3.5 cm, 2.5×4 cm, 2.5×4.5 cm, 2.5×5 cm, 3×3.5 cm, 3×4 cm, 3×4.5 cm, 3×5 cm, 3.5×4 cm, 3.5×4.5 cm, 3.5×5 cm, 4×4.5 cm, 4×5 cm, or 4.5×5 cm in size, or may be no smaller, or no larger, than 1×1 cm, 1.5×1.5 cm, 2×2 cm, 2.5×2.5 cm, 3×3 cm, 3.5×3.5 cm, 4×4 cm, 4.5×4.5 cm, 5×5 cm, 1×1.5 cm, 1×2 cm, 1×2.5 cm, 1×3 cm, 1×3.5 cm, 1×4 cm, 1×4.5 cm, 1×5 cm, 1.5×2 cm, 1.5×2.5 cm, 1.5×3 cm, 1.5×3.5 cm, 1.5×4 cm, 1.5×4.5 cm, 2×2.5 cm, 2×3 cm, 2×3.5 cm, 2×4 cm, 2×4.5 cm, 2×5 cm, 2.5×3 cm, 2.5×3.5 cm, 2.5×4 cm, 2.5×4.5 cm, 2.5×5 cm, 3×3.5 cm, 3×4 cm, 3×4.5 cm, 3×5 cm, 3.5×4 cm, 3.5×4.5 cm, 3.5×5 cm, 4×4.5 cm, 4×5 cm, or 4.5×5 cm, though the biofabric may be cut to different dimensions. Pieces of collagen biofabric that are 2×2 cm, 3×3 cm, 3×2 cm, 1×2 cm, 1×1 cm or 4×4 cm are particularly preferred. Further, the biofabric may be provided as a sheet from which an end use may cut two or more pieces, or may be provided as a roll or strip.

The collagen biofabric useful in the treatment methods of the invention may be provided to the end user either dry, or pre-wetted in a suitable physiologically-compatible, medically-useful liquid, such as a saline solution. In one embodiment, the solution comprises one or more bioactive compounds, as described in Section 4.2.2, above, without limitation. Preferably, said bioactive compound is disposed onto or within the collagen biofabric such that the majority of the bioactive compound contacts the tympanic membrane at some point during the time the collagen biofabric contacts the tympanic membrane.
4.2.4 Method of Making Collagen Biofabric

Collagen biofabric made from amniotic membrane may be produced by any means that preserves the biochemical and structural characteristics of the membrane's components—chiefly collagen, elastin, laminin, and fibronectin. A preferred material is the collagen biofabric described in, and produced according to the methods disclosed in, United States Application Publication No. U.S. 2004/0048796 A1, “Collagen Biofabric and Methods of Preparation and Use Therefor” by Hariri, which is hereby incorporated in its entirety.

Preferably, the collagen biofabric used to repair a tympanic membrane is from a human placenta for use in human subjects, though the collagen biofabric may be made from amniotic membrane from a non-human mammal. Where the collagen biofabric is to be used to treat a tympanic membrane of a non-human animal, it is preferred that the collagen biofabric used be derived from a placenta from that species of animal.

In a preferred embodiment, the placenta for use in the methods of the invention is taken as soon as possible after delivery of the newborn. The placenta may be used immediately, or may be stored for 2-5 days from the time of delivery prior to any further treatment. The placenta is typically exsanguinated, i.e., drained of the cord blood remaining after birth. Preferably, the expectant mother is screened prior to the time of birth, using standard techniques known to one skilled in the art, for communicable diseases including but not limited to, HIV, HBV, HCV, HTLV, syphilis, CMV, and other viral pathogens known to contaminate placental tissue.

One exemplary method for preparing a collagen biofabric of the invention comprises the following steps:

Step I. The umbilical cord is separated from the placental disc; optionally, the amniotic membrane is separated from the chorionic membrane. In a preferred embodiment, the amniotic membrane is separated from the chorionic membrane prior to cutting the placental membrane. Following separation of the amniotic membrane from the chorionic membrane and placental disc, the umbilical cord stump is cut, e.g., with scissors, and detached from the placental disc. The amniotic membrane may then be stored in a sterile, preferably buffered, saline solution, such as 0.9% sterile NaCl solution. Preferably, the amniotic membrane is stored by refrigeration, at a temperature of at least 2° C.

Step II. The amniotic membrane is substantially decellularized; that is, substantially all cellular material and cellular debris (e.g., all visible cellular material and cellular debris) is removed. Any decellularizing process known to one skilled in the art may be used, however, generally the process used for decellularizing the amniotic membrane of the invention does not disrupt the native conformation of the proteins making up the biofabric. “Substantial decellularization” of the amniotic membrane preferably removes at least 90% of the cells, more preferably removes at least 95% of the cells, and most preferably removes at least 99% of the cells (e.g., fibroblasts, amniocytes and chorionocytes). The amniotic membranes decellularized in accordance with the methods of the invention are uniformly thin, with variations in thickness of between about 2 and about 150 microns in the dry state, smooth (as determined by touch) and translucent. Decellularization may comprise physical scraping, for example, with a sterile cell scraper, in combination with rinsing with a sterile solution. The decellularization technique employed preferably does not result in gross disruption of the anatomy of the amniotic membrane or alter the biomechanical properties of the amniotic membrane. Preferably, the decellularization of the amniotic membrane comprises use of a detergent-containing solution, such as nonionic detergents, Triton X-100, anionic detergents, sodium dodecyl sulfate, Any mild anionic detergent, i.e., a non-caustic detergent, with a pH of 6 to 8, and low foaming, can be used to decellularize the amniotic membrane. In a specific embodiment, 0.01-10% deoxycholic acid sodium salt monohydrate is used in the decellularization of the amniotic membrane. Decellularization using enzyme solution, such as a trypsin-containing buffer, can also be used.

It is highly preferable to limit the protease activity in preparation of the biofabric. Additives to the lysis, rinse and storage solutions such as metal ion chelators, for example 1,10-phenanthroline and ethylenediaminetetraacetic acid (EDTA), create an environment unfavorable to many proteolytic enzymes. Providing sub-optimal conditions for proteases such as collagenase, assists in protecting amniotic membrane components such as collagen from degradation during the cell lysis step. Suboptimal conditions for proteases may be achieved by formulating the hypotonic lysis solution to eliminate or limit the amount of calcium and zinc ions available in solution. Many proteases are active in the presence of calcium and zinc ions and lose much of their activity in calcium and zinc ion free environments. Preferably, the hypotonic lysis solution will be prepared selecting conditions of pH, reduced availability of calcium and zinc ions, presence of metal ion chelators and the use of proteolytic inhibitors specific for collagenase such that the solution will optimally lyse the native cells while protecting the underlying amniotic membrane from adverse proteolytic degradation. For example a hypotonic lysis solution may include a buffered solution of water, pH 5.5 to 8, preferably pH 7 to 8, free from calcium and zinc ions and including a metal ion chelator such as EDTA. Additionally, control of the temperature and time parameters during the treatment of the amniotic membrane with the hypotonic lysis solution may also be employed to limit the activity of proteases.

It is preferred that the decellularization treatment of the amniotic membrane also limits the generation of new immunological sites. Since enzymatic degradation of collagen is believed to lead to heightened immunogenicity, the invention encompasses treatment of the amniotic membrane with enzymes, e.g., nucleases, that are effective in inhibiting cellular metabolism, protein production and cell division, that minimize proteolysis of the compositions of the amniotic membrane thus preserving the underlying architecture of the amniotic membrane. Examples of nucleases that can be used in accordance with the methods of the invention are those effective in digestion of native cell DNA and RNA including both exonucleases and endonucleases. A non-limiting example of nucleases that can be used in accordance with the methods of the invention include exonucleases that inhibit cellular activity, e.g., DNase I (SIGMA Chemical Company, St. Louis, Mo.) and RNase A (SIGMA Chemical Company, St. Louis, Mo.) and endonucleases that inhibit cellular activity, e.g., EcoRI (SIGMA Chemical Company, St. Louis, Mo.) and Hindlll (SIGMA Chemical Company, St. Louis, Mo.). It is preferable that the selected nucleases are applied in a physiological buffer solution which contains ions, e.g., magnesium, calcium, which are optimal for the activity of the nuclease. Preferably, the ionic concentration of the buffered solution, the treatment temperature and the length of treatment are selected by one skilled in the art by routine experimentation to assure the desired level of nuclease activity. The buffer is preferably hypotonic to promote access of the nucleases to cell interiors.

In another embodiment of Steps I and II, above, the placenta, after initial processing, is briefly rinsed in saline to remove blood from the placental surface. The placental disk is then immersed in a cold deoxycholic acid solution at a concentration of about 0.1% to about 10%, and, in a specific embodiment, about 0.1% to about 2.0%. The placenta is then incubated in this solution at between about 1° C. to about 8° C. for about 5 days to about 6 months. In specific embodiments, the placental disk is immersed, for example, for about 5 to about 15 days; about 5 to about 30 days, about 5 to about 60 days, or for up to about one year. Typically, the deoxycholic acid solution is replaced during incubation every 2-5 days. In another specific embodiment, the placental disk is immersed in a deoxycholic acid solution at a concentration of about 1% at a temperature of 0° C. to about 8° C. for about 5 days to about 15 days. This incubation serves two purposes. First, it allows time for serological tests to be performed on the placental material and blood, so that placentas failing to meet serological criteria are not processed further. Second, the longer incubation improves the removal of epithelial cells and fibroblasts, which allows for a significant reduction in the amount of time spent decellularizing the amnion by physically scraping. Typically, the scraping time is reduced from, e.g., about 40 minutes to about 20 minutes. The amniotic membrane is then dried as described below.

In one embodiment of Steps I and II, therefore, the amniotic membrane is separated from the chorion, as described above, and the amnion is rinsed briefly. The amnion is then incubated in 1% deoxycholic acid at 4° C. for 10 days, with a change of the deoxycholic acid solution on the fifth day of incubation. Serological test results are evaluated, and the amnion is either accepted or rejected in part on the results. Once incubation is complete, epithelial cells and fibroblasts still clinging to the amnion are removed by scraping. The amnion is rinsed, and then dried as described below.

Step III. Following decellularization, the amniotic membrane is washed to assure removal of detergent and, if used, enzymes used for decellularization. This process also removes cellular debris which may include cellular debris. The wash solution may be de-ionized water or an aqueous hypotonic buffer. Preferably, the amniotic membrane is gently agitated for 15-120 minutes in the detergent, e.g., on a rocking platform, to assist in the decellularization. The amniotic membrane may, after detergent decellularization, again be physically decellularized as described supra; the physical and detergent decellularization steps may be repeated as necessary, as long as the integrity of the amniotic membrane is maintained, until no visible cellular material and cellular debris remain.

In certain embodiments, the amniotic membrane is dried immediately (i.e., within 30 minutes) after the decellularization and washing steps. Alternatively, when further processing is not done immediately, the amniotic membrane may be refrigerated, e.g., stored at a temperature of about 1° C. to about 20° C., preferably from about 2° C. to about 8° C., for up to 28 days prior to drying. When the decellularized amniotic membrane is stored for more than three days but less than 28 days, the sterile solution covering the amniotic membrane is preferably changed periodically, e.g., every 1-3 days.

In certain embodiments, when the amniotic membrane is not refrigerated after washing, the amniotic membrane is washed at least 3 times prior to proceeding to Step IV of the preparation. In other embodiments, when the amniotic membrane has been refrigerated and the sterile solution has been changed once, the amniotic membrane is washed at least twice prior to proceeding to Step IV of the preparation. In yet other embodiments, when the amniotic membrane has been refrigerated and the sterile solution has been changed twice or more, the amniotic membrane is washed at least once prior to proceeding to Step IV of the preparation.

Prior to proceeding to Step IV, it is preferred that all bacteriological and serological testing be assessed to ensure that all tests are negative.

Step IV. The final step in this embodiment of the method of collagen biofabric production comprises drying the decellularized amniotic membrane of the invention to produce the collagen biofabric. Any method of drying the amniotic membrane so as to produce a flat, dry sheet of collagen may be used. Preferably, however, the amniotic membrane is dried under vacuum.

In a specific embodiment, an exemplary method for drying the decellularized amniotic membrane of the invention comprises the following steps:

Assembly of the decellularized amniotic membrane for drying. The decellularized amniotic membrane is removed from the sterile solution, and the excess fluid is gently squeezed out. The decellularized amniotic membrane is then gently stretched until it is flat with the fetal side faced in a downward position, e.g., on a tray. The decellularized amniotic membrane is then flipped over so that fetal side is facing upwards, and placed on a drying frame, preferably a plastic mesh drying frame (e.g., QUICK COUNT® Plastic Canvas, Uniek, Inc., Waunakee, Wis.). In other embodiments, the drying frame may be any autoclavable material, including but not limited to a stainless steel mesh. In a most preferred embodiment, about 0.5 centimeter of the amniotic membrane overlaps the edges of the drying frame. In certain embodiments, the overlapping amniotic membrane extending beyond the drying frame is wrapped over the top of the frame, e.g., using a clamp or a hemostat. Once the amniotic membrane is positioned on the drying frame, a sterile gauze is placed on the drying platform of a heat dryer (or gel-dryer) (e.g., Model 583, Bio-Rad Laboratories, 200 Alfred Nobel Drive, Hercules, Calif. 94547), so that an area slightly larger than the amniotic membrane resting on the plastic mesh drying frame is covered. Preferably, the total thickness of the gauze layer does not exceed the thickness of one folded 4×4 gauze. Any heat drying apparatus may be used that is suitable for drying sheet like material. The drying frame is placed on top of the gauze on the drying platform so that the edges of the plastic frame extend above beyond the gauze edges, preferably between 0.1-1.0 cm, more preferably 0.5-1.0 cm. In a most preferred embodiment, the drying frame having the amniotic membrane is placed on top of the sterile gauze with the fetal side of the amniotic membrane facing upward. In some embodiments, another plastic framing mesh is placed on top of the amniotic membrane. In another embodiments, a sheet of thin plastic (e.g., SW 182, clear PVC, AEP Industries Inc., South Hackensack, N.J. 07606) or a biocompatible silicone is placed on top of the membrane covered mesh so that the sheet extends well beyond all of the edges. In this embodiment, the second mesh frame is not needed.

In an alternative embodiment, the amniotic membrane is placed one or more sterile sheets of TYVEK® material (e.g., a sheet of TYVEK® for medical packaging, Dupont TYVEK®, P.O. Box 80705, Wilmington, Del. 19880-0705), optionally, with one sheet of TYVEK® on top of the membrane (prior to placing the plastic film). This alternate process will produce a smoother version of the biofabric (i.e., without the pattern of differential fiber compression regions along and perpendicular to the axis of the material), which may be advantageous for certain applications, such as for example for use as a matrix for expansion of cells.

Drying the amniotic membrane. In one embodiment, the invention encompasses heat drying the amniotic membrane of the invention under vacuum. While the drying under vacuum may be accomplished at any temperature from about 0° C. to about 60° C., the amniotic membrane is preferably dried at between about 35° C. and about 50° C., and most preferably at about 50° C. It should be noted that some degradation of the collagen is to be expected at temperatures above 50° C. The drying temperature is preferably set and verified using a calibrated digital thermometer using an extended probe. Preferably, the vacuum pressure is set to about −22 inches of Hg. The drying step is continued until the collagen matrix of the amniotic membrane contains less than 3-12% water as determined for example by a moisture analyzer. To accomplish this, the amniotic membrane may be heat-vacuum dried, e.g., for approximately 60 minutes to achieve a dehydrated amniotic membrane. In some embodiments, the amniotic membrane is dried for about 30 minutes to 2 hours, preferably about 60 minutes. Although not intending to be bound by any theory or mechanism of action, it is believed that the low heat setting coupled with vacuum pressure allows the amniotic membrane to achieve the dehydrated state without denaturing the collagen.

After completion of the drying process in accordance with the invention, the amniotic membrane is cooled down for approximately two minutes with the vacuum pump running.

Packaging and Storing of the Amniotic Membrane. Once the amniotic membrane is dried, the membrane is gently lifted off the drying frame. “Lifting off” the membrane may comprise the following steps: while the pump is still running, the plastic film is gently removed from the amniotic membrane starting at the corner, while holding the amniotic membrane down; the frame with the amniotic membrane is lifted off the drying platform and placed on a cutting board with the amniotic membrane side facing upward; an incision is made, cutting along the edge 1-2 mm away from the edge of the frame; and the amniotic membrane is then peeled off the frame. Preferably, handling of the amniotic membrane at this stage is done with sterile gloves.

The amniotic membrane is placed in a sterile container, e.g., a peel pouch, and is sealed. The biofabric produced in accordance with the methods of the invention may be stored at room temperature for an extended period of time as described supra. In alternative embodiments, the invention provides a method of preparing a collagen biofabric comprising a chorionic membrane, or both a chorionic membrane and an amniotic membrane. The methods described above are applicable to the method of preparing a biofabric comprising a chorionic membrane, or both a chorionic membrane and an amniotic membrane. In one embodiment, the invention encompasses the use of a collagen biofabric prepared by providing a placenta comprising an amniotic membrane and a chorionic membrane; separating the amniotic membrane from the chorionic membrane; and decellularizing the chorionic membrane. In a specific embodiment, the method further entails washing and drying the decellularized chorionic membrane. In another embodiment, the invention encompasses the use of a collagen biofabric prepared by providing a placenta comprising an amniotic membrane and a chorionic membrane, and decellularizing the amniotic and chorionic membranes. In a specific embodiment, the method further entails washing and drying the decellularized amniotic and chorionic membranes.
4.2.5 Storage and Handling of Collagen Biofabric

Dehydrated collagen biofabric may be stored, e.g., as dehydrated sheets, at room temperature (e.g., 25° C.) prior to use. In certain embodiments, the collagen biofabric can be stored at a temperature of at least 10° C., at least 15° C., at least 20° C., at least 25° C., or at least 29° C. Preferably, collagen biofabric, in dehydrated form, is not refrigerated. In some embodiments, the collagen biofabric may be refrigerated at a temperature of about 2° C. to about 8° C. The biofabric produced according to the methods of the invention can be stored at any of the specified temperatures for 12 months or more with no alteration in biochemical or structural integrity (e.g., no degradation), without any alteration of the biochemical or biophysical properties of the collagen biofabric. The biofabric can be stored for several years with no alteration in biochemical or structural integrity (e.g., no degradation), without any alteration of the biochemical or biophysical properties of the collagen biofabric. The biofabric may be stored in any container suitable for long-term storage. Preferably, the collagen biofabric of the invention is stored in a sterile double peel-pouch package.

The collagen biofabric may be hydrated prior to use. The collagen biofabric can be rehydrated using, e.g., a sterile physiological buffer. In a specific embodiment, the sterile saline solution is a 0.9% NaCl solution. In some embodiments the sterile saline solution is buffered. In certain embodiments, the hydration of the collagen biofabric of the invention requires at least 2 minutes, at least 5 minutes, at least 10 minutes, at least 15 minutes, or at least 20 minutes. In a preferred embodiment, the hydration of the collagen biofabric of the invention is complete within 5 minutes. In yet another preferred embodiment, the hydration of the collagen biofabric of the invention is complete within 10 minutes. In yet another embodiment, the hydration of the collagen biofabric of the invention takes no more than 10 minutes. Once hydrated, the collagen biofabric may be maintained in solution, e.g., sterile 0.9% NaCl solution, for up to six months, with a change of solution, e.g., every three days.
4.2.6 Sterilization

Sterilization of the biofabric may be accomplished by any medically-appropriate means, preferably means that do not significantly alter the tertiary and quaternary structure of the amniotic membrane proteins. Sterilization may be accomplished, for example, using gas, e.g., ethylene dioxide. Sterilization may be accomplished using radiation, for example, gamma radiation, and is preferably done by electron beam irradiation using methods known to one skilled in the art, e.g., Gorham, D. Byrom (ed.), 1991, Biomaterials, Stockton Press, New York, 55-122. Any dose of radiation sufficient to kill at least 99.9% of bacteria or other potentially contaminating organisms is within the scope of the invention. In a preferred embodiment, a dose of at least 18-25 kGy is used to achieve the terminal sterilization of the biofabric.
4.2.7 Laminates

The collagen biofabric may be laminated to provide greater stiffness and durability during the healing process (typically about three months). The collagen biofabric may be laminated as follows.

Collagen biofabric is typically laminated by stacking 2 or more layers of collagen biofabric one atop the other and sealing or drying. The collagen biofabric may be laminated either dry or after rehydration. Alternatively, two or more layers of, e.g., amniotic membrane may be laminated prior to initial drying after cell removal, e.g., via a cell scraping step (see Examples, below). If laminated prior to the initial drying, 2 or more collagen biofabric layers may be stacked one atop the other and subsequently dried, using, for example, a freeze-drying process, or drying under moderate heat with or without vacuum. The heat applied preferably is not so intense as to cause breakdown or decomposition of the protein components, especially the collagen, of the collagen biofabric. Typically, the heat applied is no more than about 70° C., preferably no more than about 60° C., and, more preferably, is approximately 50° C. Lamination time varies with, e.g., the number of layers being laminated, but typically takes 1-2 hours at 50° C. for the size pieces of collagen biofabric used for tympanic membrane repair. Preferably, the collagen biofabric laminate comprises 2-6 layers of collagen biofabric. In one preferred embodiment, the collagen biofabric laminate has two layers and is approximately 50 micrometers in thickness. In another embodiment, the collagen biofabric laminate has two layers and has a thickness of about 20-60 microns. Preferably, each of the layers is from the same collagen biofabric lot, that is, the same placenta.

The collagen biofabric may also, for example, be laminated using an adhesive applied between 2 or more layers of collagen biofabric or amniotic membrane. Such an adhesive is preferably appropriate for medical applications, and can comprise, for example, a natural biological adhesive, for example fibrin glue, a synthetic adhesive, or combinations thereof. The adhesive may further be chemically converted from precursors during the lamination process.
4.2.8 Stem Cells

The tympanic membrane repair methods, as well as the collagen biofabric used in the treatment methods, as described herein, may also comprise stem or progenitor cells. Preferably, the treatment method comprises the use of stem or progenitor cells to encourage tympanic membrane regrowth. Preferably, the collagen biofabric comprises mesenchymal or mesenchymal-like stem cells, for example, those described in U.S. Pat. Nos. 5,486,359, 6,261,549 and 6,387,367, or placenta-derived stem cells such as those described in U.S. Application Publication Nos. 2002/0123141, 2003/0032179 and 2003/0180269. However, the collagen biofabric may comprise stem or progenitor cells, preferably mammalian stem or progenitor cells, from any tissue source. The collagen biofabric may comprise embryonic stem cells or embryonic germ cells.

The combination of collagen biofabric and stem or progenitor cells may be accomplished prior to or during application of the collagen biofabric to a tympanic membrane. For example, a sheet or piece of collagen biofabric may be prepared immediately prior to application on the tympanic membrane by disposing on the surface of the collagen biofabric a solution of stem or progenitor cells and allowing the stem or progenitor cells sufficient time to attach to the collagen biofabric. The stem or progenitor cells, alternately, may be disposed onto the surface of the collagen biofabric about 30 minutes, 1, 2, 3,4, 5, 6, 7, 8, 9, 10, 12, 14, 16, 18, 10, 12, 24 or more hours prior to application of the collagen biofabric onto the tympanic membrane. The number of stem or progenitor cells disposed onto the surface of the collagen biofabric may vary, but may be at least 1×10⁶, 3×10⁶, 1×10⁷, 3×10⁷, 1×10⁸, 3×10⁸, 1×10⁹, 3×10⁹, 1×10¹⁰, 3×10¹⁰, 1×10¹¹, 3×10¹¹, or 1×10¹²; or may be no more than 1×10⁶, 3×10⁶, 1×10⁷, 3×10⁷, 1×10⁸, 3×10⁸, 1×10⁹, 3×10⁹, 1×10¹⁰, 3×10¹⁰, 1×10¹¹, 3×10¹¹, or 1×10¹² stem or progenitor cells. Alternatively, in another embodiment, the stem or progenitor cells, in the number indicated above, may be disposed on the surface of the collagen biofabric after the collagen biofabric has been applied to a tympanic membrane. In another embodiment, the stem cells are applied directly to the tympanic membrane in any of the amounts indicated above, and the tympanic membrane is covered with the collagen biofabric. In a more specific embodiment, the stem cells are applied in a physiologically-acceptable liquid, such as a saline solution, or embedded in a physiologically-acceptable gel, such as a hydrogel, in which the stem or progenitor cells may be maintained and migrate through. The stem cells, prior to or after contacting with a tympanic membrane, may be contacted with one or more differentiation-modulating agents, for example, the differentiation-modulating agents described in U.S. Application Publication Nos. 2003/0235909, 2004/0028660, or International Application Publication No. WO 03/087333. Methods of differentiating stem cells to, for example, epidermal, mesodermal, and other cell types are known in the art, and are described, e.g., in U.S. Application Publication No. 2004/0028660.

4.3 Kits

Collagen biofabric, useful for the methods of tympanic membrane repair of the present invention may be provided in a wrapping or container as part of a kit for the facilitation of the repair of a tympanic membrane. In a specific embodiment, the collagen biofabric is provided an a sterile double-peel package. In a more specific embodiment, the collagen biofabric is about 6×8 cm. The kit may comprise one or more pieces of collagen biofabric and any other medical device, disposable or drug that would facilitate repair of a tympanic membrane. Preferably, each piece of the collagen biofabric in the kit is provided as a single sheet or patch in a sterile container or wrapping separate from the remainder of kit contents. In another embodiment, the kit comprises two or more pieces of collagen biofabric, separately wrapped or contained. In another embodiment, said kit comprises a support for the collagen biofabric. In specific embodiments, the support may be a natural or a synthetic material. In other specific embodiments, said support is a plastic film, plastic sheet, or a stretchable plastic wrap. In another embodiment, said kit comprises one or more disposables. In a specific embodiment, said disposables are bandages, means for sterilizing the skin surrounding a tympanic membrane, swabs, gloves, or sterile sheets. In another embodiment, said kit comprises an antibiotic ointment, cream, or spray. In another embodiment, said kit comprises a piece of collagen biofabric and one or more wound healing agents. In a specific embodiment, said wound healing agent is PDGF, TGF, hyaluronic acid, fibrin, or fibronectin.

### 5. EXAMPLES

### 5.1 Example 1

### Method of Making Collagen Biofabric

Materials

The following materials were used in preparation of the collagen biofabric.

Materials/Equipment

Copy of Delivery Record
Copy of Material/Family Health History/Informed Consent
Source Bar Code Label (Donor ID number)
Collection # (A sequential number is assigned to incoming material)
Tissue Processing Record (Document ID #ANT-19F); a detailed record of processing of each lot number is maintained
Human Placenta (less than 48 hours old at the start of processing)
Sterile Surgical Clamps/Hemostats
Sterile Scissors
Sterile Scalpels
Sterile Steri-Wipes
Sterile Cell Scraper (Nalgene NUNC Int. R0896)
Sterile Gauze (non-sterile PSS 4416, sterilized)
Sterile Rinsing Stainless Steel Trays
Disinfected Processing Stainless Steel Trays
Disinfected Plastic Bin
Sterile 0.9% NaCl Solution (Baxter 2F7124)
Sterile Water (Milli Q plus 09195 or Baxter 2F7113)
Sterile Specimen Containers (VWR 15704-014)
Personal Protective Equipment (including sterile and non-sterile gloves)
Certified Clean Room
Previously Prepared Decellularizing Solution (D-cell); 0.01-1% deoxycholic acid sodium monohydrate
Disinfected Bin
Rocking Platform (VWR Model 100)
Timer (VWR 21376890)
Disinfected Plastic Frame Mesh
PVC Wrap Film
Vacuum Pump (Schuco-Vac 5711-130)
Gel Dryer (i.e., heat dryer; BioRad Model 583)
Disinfected Stainless Steel Cutting Board
Pouches for Packaging
Sterile Stainless Steel Ruler (General Tools MFG. Co 1201)
Traceable Digital Thermometer (Model 61161-364, Control Company)
Accu-Seal Automatic Sealer (Accu-Seal, Model 630-1B6)

The expectant mother was screened at the time of birth for communicable diseases such as HIV, HBV, HCV, HTLV, syphilis, CMV and other viral and bacterial pathogens that could contaminate the placental tissues being collected. Only tissues collected from donors whose mothers tested negative or non-reactive to the above-mentioned pathogens were used to produce the collagen biofabric.

Following normal birth, the placenta, umbilical cord and umbilical cord blood were spontaneously expelled from the contracting uterus. The placenta, umbilical cord, and umbilical cord blood were collected following birth. The materials were transported to the laboratory where they were processed under aseptic conditions in a Clean room having a HEPA filtration system, which was turned on at least one hour prior to processing. Gloves (sterile or non-sterile, as appropriate) were worn at all times while handling the product. All unused (waste) segments of the amnion/chorion and contaminated liquids generated during tissue processing were disposed of as soon as feasible.

Step I.

A sterile field was set up with sterile Steri-Wrap sheets and the following instruments and accessories for processing were placed on it.
sterile tray pack
sterile Cell Scraper
sterile scalpel
disinfected processing tray

Sterile pack ID # was recorded in the Processing Record.

The placenta was removed from the transport container and placed onto the disinfected stainless steel tray. Using surgical clamps and scissors, the umbilical cord was cut off approximately 2 inches from the placental disc. The umbilical cord was placed into a separate sterile container for further processing. The container was labeled with Tissue ID Bar Code; and the material and storage solution(s) present (e.g., type of media) were identified. In some cases, the umbilical cord was discarded if not requested for other projects.

Starting from the edge of the placental membrane, the amnion was separated from the chorion using blunt dissection with fingers. This was done prior to cutting the membrane.

After the amnion was separated from the entire surface of the chorion and placental disc, the amniotic membrane was cut around the umbilical cord stump with scissors and detached from the placental disc. In some instances, if the separation of the amnion and chorion was not possible without tearing the tissue, the amnion and chorion were cut from the placental disc as one piece and then peeled apart.

The chorion was placed into a separate specimen container to be utilized for other projects. The container was labeled with the Tissue ID Bar Code, the material and storage solution(s) present (e.g., type of media) were identified, initialed and dated.

If any piece of amnion was still attached to the placental disc it was peeled from the disc and cutting off around the umbilical cord with scissors. The placenta was placed back into the transport container to be utilized for other projects.

The appropriate data was recorded in the Tissue Processing Record.

The amniotic membrane was kept in the tray with sterile 0.9% NaCl solution. Preferably, the amniotic membrane is stored by refrigeration for a maximum of 72 hours from the time of delivery prior to the next step in the process.

Step II.

The amniotic membrane was removed from the specimen container one piece at a time and placed onto the disinfected stainless steel tray. Other pieces were placed into a separate sterile stainless steel tray filled with sterile water until they were ready to be cleaned. Extra pieces of amnion from the processing tray were removed and placed in a separate rinsing stainless steel tray filled with sterile water.

The amniotic membrane was rinsed with sterile water if grossly contaminated with blood maternal or fetal fluids/materials changing sterile water as needed.

The amniotic membrane was placed on the processing tray with the maternal side facing upward. Using a sterile Cell Scraper, as much as possible of visible contamination and cellular material from the maternal side of the amnion was carefully removed. (Note: minimal pressure should be applied for this step to prevent tearing the membrane). Sterile water was used to aid in the removal of cells and cellular debris. The amniotic membrane was further rinsed with sterile water in the separate sterile stainless steel rinsing tray.

The amniotic membrane was turned over so that the fetal side was facing upward and placed back on the processing tray and rinsed with sterile water. Visible cellular material and debris using the Cell Scraper was gently removed (Note: minimal pressure should be applied for this step to prevent tearing the membrane). Sterile water was used to aid in the removal of cells and cellular debris.

The amniotic membrane was rinsed with sterile water in between cleaning rounds in separate sterile rinsing trays. The tissue was cleaned as many times (cleaning rounds) as necessary to remove most if not all of visible cellular material and debris from both sides of the membrane. The sterile water was changed in the rinsing trays in between rinses.

The processing tray was rinsed with sterile water after each cleaning round.

All other pieces of amnion were processed in the same manner and placed into the same container. Tissue Id Bar Code was affixed, the material and storage solution(s) present (e.g., type of media) were identified, initials date were added.

The appropriate information and the date were recorded in the Tissue Processing Record.

Step III.

The amniotic membrane was removed from the rinsing tray, (or from storage container) excess fluid was gently squeezed out with fingers and the membrane was placed into the sterile specimen container. The container was filled up to the 150 ml mark with D-cell solution ensuring that all of the amniotic membrane was covered and the container was closed.

The container was placed in the bin on the rocking platform. The rocking platform was turned on and the membrane was agitated in D-cell solution for a minimum of 15 minutes and a maximum of 120 minutes at Setting #6.

A new sterile field was set up with new sterile instruments and disinfected tray in a same manner as in the Step I. Sterile pack ID # was recorded in the Processing Record.

After agitation was completed, the rocking platform was turned off and the membrane was removed from the container. The membrane was placed into a new sterile stainless steel processing tray. Sterile 0.9% NaCl solution was added to cover the bottom of the tray.

Using a new sterile Cell Scraper, residual D-cell and cellular material (if any) was removed from both sides of the tissue. This step was repeated as many times as needed to remove as much as possible of visible residual cellular material from the entire surface on both sides. The membrane was rinsed with sterile 0.9% NaCl solution in a separate rinsing tray in between cleaning rounds. The sterile 0.9% NaCl solution was changed in the rinsing trays in between rinses.

After the last cleaning round was completed, the membrane was rinsed with sterile 0.9% NaCl solution and placed into the new sterile specimen container filled with sterile 0.9% NaCl solution.

All remaining pieces of amniotic membrane were processed in exactly the same manner.

When all amniotic membrane pieces were processed and in the container with the sterile 0.9% NaCl solution, the container was placed in the bin on the rocking platform to agitate for a minimum of 5 minutes at setting #6. After agitation was completed, the membrane was removed from the specimen container, the sterile 0.9% NaCl solution was changed in the container and the membrane was placed back into the specimen container.

The specimen container was labeled with Tissue ID Bar Code and Quarantine label. The material and storage solution(s) present (e.g., type of media) were identified, initialed and dated. The specimen container was placed into a clean zip-lock bag and placed in the refrigerator (2-8° C.).

All appropriate data was recorded in the Tissue Processing Record.

When serology results became available, the appropriate label (Serology Negative or For Research Use Only) was placed on the top of the Quarantine label and those containers were segregated from Quarantined ones.

Step IV.

Before proceeding with Step IV, the Tissue Status Review was checked to make sure all applicable test results were negative.

A sterile field was set up with sterile Steri-Wrap sheet and all sterile and disinfected instruments and accessories were set up in the same manner as in Steps II and III.

The membrane was removed from the refrigerator and placed into a new sterile stainless steel processing tray. Sterile 0.9% NaCl solution was added to cover the bottom of the tray.

All visible cellular material and debris (if any) was gently removed using a new sterile Cell Scraper (Note: minimal pressure should be applied for this step to prevent tearing the membrane). Sterile 0.9% NaCl solution was used to aid in removal of the cells and debris.

The membrane was rinsed in the separate sterile stainless steel rinsing tray filled with the sterile 0.9% NaCl Solution. 0.9% NaCl Solution was changed in between cleaning rounds. The membrane was placed into a new sterile specimen container, the container was filled with fresh sterile 0.9% NaCl solution and placed on the rocking platform for agitation for a minimum of 5 minutes at Setting #6.

The previous step was repeated 3 times and the sterile 0.9% NaCl solution was changed in between each agitation. Appropriate data was recorded in the Tissue Processing Record.

The membrane was removed from the specimen container one piece at a time, excess fluid was gently squeezed out with fingers and the membrane was placed onto a sterile processing tray. The membrane was gently stretched until flat; ensuring it was fetal side down.

The frame was prepared by cutting the disinfected plastic sheet with sterile scissors. The size of the frame should be approximately 0.5 cm smaller in each direction than the membrane segment. The frame was rinsed in the rinsing tray filled with sterile 0.9% NaCl solution.

The frame was placed on the slightly stretched membrane surface and pressed on it gently. It is imperative that the smooth side of the plastic frame faces the tissue.

Using a scalpel, the membrane was cut around the frame leaving approximately 0.5 cm extending beyond frame edges. The excess membrane was placed back into the specimen container

The membrane edges that are extended beyond the frame were wrapped over the edges of the frame using clamps or tweezers and put aside on the same tray.

The next piece of membrane was processed in the same manner. It is important the total area to be dried does not exceed 300 cm² per heat dryer. While ‘framing out’ the piece of membrane, the non-framed pieces should remain in the container in sterile 0.9% NaCl solution.

The drying temperatures of dryers were set and verified using a calibrated digital thermometer with extended probe. The drying temperature was set at 50° C. The data was recorded in the Tissue Processing Record.

The vacuum pump was turned on.

A sterile gauze was placed on the drying platform of the heat dryer, covering an area slightly larger than the area of the framed membrane. It is important to make sure that the total thickness of the gauze layer does not exceed thickness of one folded 4×4 gauze.

One sheet of plastic framing mesh was placed on top of the gauze. The plastic mesh edges should extend approximately 0.5-1.0 cm beyond gauze edges.

The framed membrane was gently lifted and placed on the heat dryer platform on top of the plastic mesh with the membrane side facing upward. This was repeated until the maximum amount of membrane (without exceeding 300 cm²) was on the heat dryer platform. (NOTE: fetal side of the amnion is facing up).

A piece of PVC wrap film was cut large enough to cover the entire drying platform of the heat dryer plus an extra foot.

With the vacuum pump running, the entire drying platform of the heat dryer was gently covered with the plastic film leaving ½ foot extending beyond drying platform edges on both sides. Care was taken that the film pull tightly against the membrane and frame sheet (i.e., it is “sucked in” by the vacuum) and that there were no air leaks and no wrinkles over the tissue area). The lid was subsequently closed.

The vacuum pump was set to approximately −22 inches Hg of vacuum. The pump gage was recorded after 2-3 min of drying cycle. The membrane was heat vacuum dried for approximately 60 minutes. Approximately 15-30 minutes into the drying process, the sterile gauze layer was replaced in the heat dryer with a new one. The total thickness of the gauze layer must not exceed thickness of one folded 4×4 gauze.

After the change, care was taken so that the plastic film pulled tightly against the membrane and the frame sheet and there were no air leaks and no wrinkles over the membrane area.

The integrity of the vacuum seal was periodically checked by checking the pump pressure manometer. After completion of the drying process, the heat dryer was opened and the membrane was cooled down for approximately two minutes with the pump running.

A new sterile field was set up with sterile Steri-wrap and disinfected stainless steel cutting board underneath it. As this point sterile gloves were used. With the pump still running, the plastic film was gently removed from the membrane sheet starting at the corner and holding the membrane sheet down with a gloved hand. The frame was gently lifted with the membrane off the drying platform and placed on the sterile field on the top of the disinfected stainless steel cutting board with the membrane side facing upward. Using a scalpel, the membrane sheet was cut through making an incision along the edge 1-2 mm away from the edge of the frame. The membrane was held in place with a gloved (sterile glove) hand. Gently the membrane sheet was lifted off of the frame by peeling it off slowly and then placed on the sterile field on the cutting board.

Using scalpel or sharp scissors, the membrane sheet was cut into segments of specified size. All pieces were cut and secured on the sterile field before packaging. A single piece of membrane was placed inside the inner peel-pouch package with one hand (sterile) while holding the pouch with another hand (non-sterile). Care was taken not to touch pouches with “sterile” hand. After all pieces were inside the inner pouches they were sealed. A label was affixed with the appropriate information (e.g., Part #, Lot #, etc.) in the designated area on the outside of the pouch. All pieces of membrane were processed in the same manner. The labeled and sealed peel-pouch packages were placed in the waterproof zip-lock bag for storage until they were ready to be shipped to the sterilization facility or distributor. All appropriate data were recorded on the Tissue Processing Record.

### 5.2 Example 2

### Alternative Method of Making Collagen Biofabric

A placenta is prepared substantially as described in Step I of Example 1 using the Materials in that Example. An expectant mother is screened at the time of birth for communicable diseases such as HIV, HBV, HCV, HTLV, syphilis, CMV and other viral and bacterial pathogens that could contaminate the placental tissues being collected. Only tissues collected from donors whose mothers tested negative or non-reactive to the above-mentioned pathogens are used to produce the collagen biofabric.

A sterile field is set up with sterile Steri-Wrap sheets and the following instruments and accessories for processing were placed on it: sterile tray pack; rinsing tray, stainless steel cup, clamp/hemostats, tweezers, scissors, gauze.

The placenta is removed from the transport container and placed onto a disinfected stainless steel tray. Using surgical clamps and scissors, the umbilical cord is cut off approximately 2 inches from the placental disc.

Starting from the edge of the placental membrane, the amnion is separated from the chorion using blunt dissection with fingers. This is done prior to cutting the membrane. After the amnion is separated from the entire surface of the chorion and placental disc, the amniotic membrane is cut around the umbilical cord stump with scissors and detached from the placental disc. In some instances, if the separation of the amnion and chorion is not possible without tearing the tissue, the amnion and chorion is cut from the placental disc as one piece and then peeled apart.

The appropriate data is recorded in the Tissue Processing Record.

The amniotic membrane is rinsed with sterile 0.9% NaCl solution to remove blood and fetal fluid or materials. The saline solution is replaced as necessary during this rinse.

The amnion is then placed in a 0.9% saline, 1.0% deoxycholic acid solution in a specimen container and refrigerated at 2-8° C. for up to 15 days, with changes of the solution every 3-5 days. During or at the end of incubation, the serological tests noted above are evaluated. If the tests indicate contamination with one or more pathogens, the amnion is rejected and processed no further. Tissue indicated as derived from a CMV-positive donor, however, is still suitable for production of biofabric.

Once the incubation is complete, the amnion is removed from the specimen container, placed in a sterile tray and rinsed three times with 0.9% NaCl solution to reduce the deoxycholic acid from the tissue. With the amnion placed maternal side up, the amnion is gently scraped with a cell scraper to remove as much cellular material as possible. Additional saline is added as needed to aid in the removal of cells and cellular debris. This step is repeated for the fetal side of the amnion. Scraping is followed by rinsing, and is repeated, both sides, as many times as necessary to remove cells and cellular material. The scraped amnion is rinsed by placing the amnion in 0.9% saline solution a separate container on a rocking platform for 5-120 minutes at setting #6. The saline solution is replaced, and the rocking rinse is repeated.

After rinsing is complete, the amnion is optionally stored in a zip-lock bag in a refrigerator.

The scraped amnion is then placed fetal side down onto a sterile processing tray. The amnion is gently massaged by hand to remove excess liquid, and to flatten the membrane. A sterile plastic sheet is cut so that its dimensions are approximately 0.5 cm smaller in each direction than the flat amnion. This plastic sheet is briefly rinsed in 0.9% NaCl solution. The plastic sheet is placed, smooth side down, on the flattened amnion, leaving a margin of uncovered amnion. A scalpel is used to trim the amnion, leaving approximately 0.5 cm extending beyond the sheet edges. These extending amnion edges are wrapped back over the plastic sheet. The total tissue area to be dried does not exceed 300 cm² for a standard vacuum heat dryer.

A sheet of sterile gauze is placed in a vacuum heat dryer. A thin plastic mesh is placed on the gauze so that approximately 0.5-10.0 cm extends beyond the edges of the gauze. The amnion and plastic sheet are then placed into the vacuum heat dryer on top of the mesh, tissue side up, and the amnion is covered with a sheet of PVC wrap film. The dryer is set at 50° C., and the temperature is checked periodically to ensure maintenance of 50° C.±1° C. The vacuum pump is then turned on and set to approximately −22 inches Hg vacuum. Drying is allowed to proceed for 60 minutes.

The dried amnion is then stored in a sealed plastic container for further use.

### 5.3 Example 3

### Myringoplasty Using Collagen Biofabric

A patient presents with hearing loss, and bone conductance greater than air conductance. Visual inspection of the tympanic membrane reveals a marginal hole comprising about 40% of the area of the membrane, caused by a cotton swab placed too far into the auditory canal. The area of the tympanic membrane is estimated, and a piece of collagen biofabric laminate is trimmed from a 2×2 cm square of the biofabric, in the approximate shape of the tympanic membrane. The collagen biofabric laminate comprises five layers of collagen biofabric from the same lot, that is, the same original placenta. The trimmed collagen biofabric laminate is placed, via the auditory canal, against the tympanic membrane over the area of perforation in which the edges were freshly debrided and potentially oozing and gently pressed into place. The tacky nature of the exudate contributes to biomaterial adherence to the membrane. The ear is temporarily filled with gelfoam to secure the collagen biofabric laminate against the tympanic membrane.

### 5.4 Example 4

### Myringoplasty Using Collagen Biofabric

A patient presents with hearing loss, and bone conductance greater than air conductance. Visual inspection of the tympanic membrane reveals a marginal hole comprising about 40% of the area of the membrane, caused by a prior infection. A postauricular incision is made approximately 1 cm behind the postauricular crease. A T-shaped incision is made in the periosteum overlying the mastoid. The periosteum is elevated and moved anteriorly into the ear canal. The canal skin and periosteum is elevated using a duckbill elevator or round knife. A self-retaining retractor is placed to retract the canal skin and the ear forward. The canal incision is designed to create a laterally based canal skin flap or vascular strip. The horizontal incision is cut first approximately 2 to 5 mm lateral to the annulus from the 12 to the 8 o'clock position (right ear). The vertical incisions are made next. A flap is elevated anteriorly until the perforation is reached. The Eustachian tube and middle ear are then packed with gelfoam. Collagen biofabric laminate is then shaped to the proper size needed for the perforation. It is then placed into position under the anterior tympanic membrane remnant and onto the posterior canal wall. The annulus is then placed back into position posteriorly and the vascular strip is carefully moved into its anatomic place. Gelfoam is placed over the drum remnant, graft, and vascular strip and the external canal is filled with antibiotic ointment. The postauricular incision is closed subcutaneously with absorbable suture, and a mastoid dressing is applied to provide light pressure and protection.

### 5.5 Example 5

### Collagen Biofabric Laminate

The collagen biofabric produced by the methods described above was laminated as follows. Dry collagen biofabric was, in some instances, rehydrated in sterile 0.9% NaCl solution for 1 hour, 10 minutes to 1 hour, 30 minutes. Dry collagen biofabric was produced by the entire procedure outlined above (Example 1), then laminated; wet collagen biofabric was prepared up to Step III, then laminated. After mounting frames were cut, the rehydrated tissue was mounted by placing the fetal side down, placing the mounting frame on top of the tissue, and cutting the tissue, leaving about 1 cm edge around the frame. The 1 cm edge was folded over the edge of the frame using a cell scraper. These steps were repeated for adding additional pieces of wet collagen biofabric. The laminated biofabric was then placed in a gel dryer and dried to substantial dryness (<20% water content by weight). Laminates were then cut to 2×6 cm samples.

Separate lots of the laminated collagen biofabric were evaluated as follows. Dimensions of dry (DT) and wet (WT) laminated collagen biofabric were determined for laminates containing 2, 3, 5 or 8 layers, as shown in Table 1:

TABLE I

| | Thickness (μm) | Length (mm) | Width (mm) | Weight (mg) |
|---|---|---|---|---|
| DT2 | 29 ± 12 | 20.0 ± 0.3 | 5.2 ± 0.1 | 0.87 ± 0.02 |
| DT3 | 32 ± 2 | 20.5 ± 0.1 | 5.2 ± 0.2 | 1.26 ± 0.11 |
| WT2 | 20 ± 15 | 20.2 ± 0.2 | 5.0 ± 0.3 | 0.93 ± 0.17 |
| WT3 | 15 ± 5 | 19.6 ± 0.1 | 5.1 ± 0.3 | 0.9 ± 0.04 |
| WT5 | 31 ± 5 | 19.8 ± 0.4 | 5.3 ± 0.1 | 2.06 ± 0.2 |
| WT8 | 115 ± 26 | 20.3 ± 0.2 | 5.1 ± 0.4 | 4.92 ± 0.56 |

Specimens showed no signs of delamination over the first two days post-lamination, when kept under dry conditions at room temperature. The laminated collagen biofabric additionally showed no signs of delamination when kept in stirred 0.9% saline, room temperature, for ten days.

Larger laminated collagen biofabric specimens were tested for laminate durability and resistance to delamination. 1×2 cm specimens from the list listed above (i.e., DT2, DT3, WT2, WT3, WT5 and WT8) were placed in Petri dishes in 5 ml phosphate buffered saline. The specimens were left on an orbital shaker for approximately 24 hours at 95 RPM. No delamination of the specimens was observed, either during shaking or thereafter during simple handling.

Equivalents:

The present invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

What is claimed is:

1. A method of repairing a tympanic membrane having a deformity, comprising contacting said tympanic membrane with a collagen biofabric.

2. The method of claim 1, wherein said deformity is a perforation.

3. The method of claim 1, wherein said deformity is an atelectatic tympanic membrane, a deformity relating to a choleastoma, a retraction pocket or a deformity resulting from a tympanosclerosis.

4. The method of claim 2, wherein said perforation is caused by trauma.

5. The method of claim 2, wherein said perforation is caused as part of a surgical procedure.

6. The method of claim 2, wherein said contacting is sufficient to occlude the perforation.

7. The method of claim 2, wherein said perforation has not healed spontaneously within two months of developing the perforation.

8. The method of claim 1, wherein the collagen biofabric is a single layer of amniotic membrane.

9. The method of claim 1, wherein the collagen biofabric is a laminate of two or more layers of amniotic membrane.

10. The method of claim 11, wherein said laminate has two layers and is about 20 to about 60 microns in thickness.

11. The method of claim 12 wherein said laminate is about 50 microns in thickness.

12. The method of claim 2, wherein the collagen biofabric comprises less than about 20% water by weight when contacted with the tympanic membrane.

13. The method of claim 2, wherein the collagen biofabric is hydrated prior to contacting with the tympanic membrane.

14. The method of claim 1, wherein said collagen biofabric is about 2 microns to about 150 microns in thickness.

15. The method of claim 20, wherein said collagen biofabric is about 1 to about 40 microns in thickness.

16. The method of claim 1, wherein said collagen biofabric is provided in a double peel pouch.

17. Collagen biofabric, wherein the collagen biofabric is a laminate having two layers or more.

18. The collagen biofabric of claim 23 wherein said collagen biofabric is a laminate of two layers and which has an average thickness of about 20 to about 60 microns.

19. The collagen biofabric of claim 24 wherein said collagen biofabric has an average thickness of about 50 microns.

20. The collagen biofabric of claim 23 measuring about 3×3 cm or less.
